SEQUENCE LISTING (14 sequences)

SEQ ID NO: 1 - DNA, 1893 bp, synthetic construct
  misc_feature 1..1893: note = Synthetic
  source 1..1893: mol_type = other DNA
taatacgactcactatagggtcgacgtcgacgggaagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccatgatctgaaaccaactttattactatattccccacaacccccagtactttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggttactagtgataatagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagggatccagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagggaattc

SEQ ID NO: 2 - DNA, 1933 bp, synthetic construct
  misc_feature 1..1933: note = Synthetic
  source 1..1933: mol_type = other DNA
taatacgactcactatagggtcgacaaaaaaaaaaaaaaaaaaaaaaaaaaaaaaaaaaaaaaaagtcgacgggaagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccatgatctgaaaccaactttattactatattccccacaacccccagtactttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggttactagtgataatagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagggatccagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagggaattc

SEQ ID NO: 3 - DNA, 1973 bp, synthetic construct
  misc_feature 1..1973: note = Synthetic
  source 1..1973: mol_type = other DNA
taatacgactcactatagggtcgacgtcgacgggaagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccatgatctgaaaccaactttattactatattccccacaacccccagtactttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggttactagtgataatagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagggatccagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagggaattcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 2013 bp, synthetic construct
  misc_feature 1..2013: note = Synthetic
  source 1..2013: mol_type = other DNA
taatacgactcactatagggtcgacaaaaaaaaaaaaaaaaaaaaaaaaaaaaaaaaaaaaaaaagtcgacgggaagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccatgatctgaaaccaactttattactatattccccacaacccccagtactttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggttactagtgataatagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagggatccagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagggaattcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 40 bp, synthetic construct
  misc_feature 1..40: note = Synthetic
  source 1..40: mol_type = other DNA
aaaaaaaaaaaaaaaaaaaaaaaaaaaaaaaaaaaaaaaa

SEQ ID NO: 6 - DNA, 34 bp, synthetic construct
  misc_feature 1..34: note = Synthetic
  source 1..34: mol_type = other DNA
gggaagaccctcgaccgtcgattgtccactggtc

SEQ ID NO: 7 - DNA, 182 bp, synthetic construct
  misc_feature 1..182: note = Synthetic
  source 1..182: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 8 - DNA, 42 bp, synthetic construct
  misc_feature 1..42: note = Synthetic
  source 1..42: mol_type = other DNA
tgatctgaaaccaactttattactatattccccacaaccccc

SEQ ID NO: 9 - DNA, 678 bp, synthetic construct
  source 1..678: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtt

SEQ ID NO: 10 - DNA, 720 bp, synthetic construct
  misc_feature 1..720: note = Synthetic
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 11 - skipped

SEQ ID NO: 12 - DNA, 131 bp, synthetic construct
  misc_feature 1..131: note = Synthetic
  source 1..131: mol_type = other DNA
agacgctacggacttaaataattgagccttaaagaagaaattctttaagtggatgctctcaaactcagggaaacctaaatctagttatagacaaggcaatcctgagccaagccgaagtagtaattagtaag

SEQ ID NO: 13 - DNA, 36 bp, synthetic construct
  misc_feature 1..36: note = Synthetic
  source 1..36: mol_type = other DNA
accagtggacaatcgacggataacagcatatctagg

SEQ ID NO: 14 - DNA, 80 bp, synthetic construct
  misc_feature 1..80: note = Synthetic
  source 1..80: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0146005 A1
(43) Pub. Date: May 8, 2025

(54) METHODS OF MAKING CIRCULAR RNA

(71) Applicant: INNOFORCE PHARMACEUTICALS, Hangzhou, Zhejiang (CN)

(72) Inventors: Shanshan WANG, Hangzhou, Zhejiang (CN); Guo-An WANG, Hangzhou, Zhejiang (CN)

(21) Appl. No.: 18/867,800

(22) PCT Filed: May 19, 2023
(86) PCT No.: PCT/CN2023/095294

§ 371 Date: Nov. 20, 2024

(30) Foreign Application Priority Data
May 20, 2022 (WO) PCT/CN2022/094157

(51) Int. Cl. C12N 15/64; C12N 15/10; C12N 15/113

(52) U.S. Cl. CPC C12N 15/64; C12N 15/1006; C12N 15/113; C12N 2310/122; C12N 2310/532; C12N 2800/50; C12N 2830/50

(57) ABSTRACT

Provided are novel DNA constructs or RNA transcripts for making circular RNAs, as well as novel methods of producing circular RNAs.

### FIELD OF THE INVENTION

The present disclosure generally relates to novel DNA constructs or RNA transcripts for making circular RNAs, as well as novel methods of producing circular RNAs.

### BACKGROUND

Circular RNA (or circRNA) found in nature is long, noncoding RNA molecule that forms a covalently closed continuous loop without 5′-3′ polarity. Circular RNA was initially reported as a viroid consisting of a covalently closed circular RNA molecule, which was pathogenic to particular higher plants (Liu L, Wang J, Khanabdali R et al (2017) Circular RNAs: isolation, characterization and their potential role in diseases. RNA Biol 14 (12): 1715-1721). Additional types of circular RNAs in many species have been described thereafter, including the circular single-stranded RNA genome of the hepatitis delta virus (HDV), or circular RNAs as products or intermediates of tRNA and rRNA maturation in archaea. Circular RNAs are generally formed by covalent binding of the 5′ site of an upstream exon with the 3′ site of the same or a downstream exon. Two different models of circular RNA biogenesis have been described, the lariat or exon skipping model and the direct backsplicing model. In the lariat model, canonical splicing occurs before backsplicing, whereas in the direct backsplicing model, the circular RNA is generated first (Jeck W R, Sorrentino J A, Wang K et al (2013) Circular RNAs are abundant, conserved, and associated with ALU repeats. RNA 19:141-157; Barrett S P, Wang P L, Salzman J (2015) Circular RNA biogenesis can proceed through an exon-containing lariat precursor. Elife 4: e07540).

Circular RNA regulates gene expression by modulating microRNAs and functions as potential biomarker. Circular RNAs can be translated in vivo to link between their expression and disease (Li J, Yang J, Zhou P, et al (2015a) Circular RNAs in cancer: novel insights into origins, properties, functions and implications. Am J Cancer Res 5 (2): 472; Li P, Chen S, Chen H et al (2015b) Using circular RNA as a novel type of biomarker in the screening of gastric cancer. Clin Chim Acta 444:132-136; Greene J, Baird A M, Brady L et al (2017) Circular RNAs: biogenesis, function and role in human diseases. Front Mol Biosci 4:38). They are resistant to RNA exonuclease and can convert to the linear RNA (by microRNA) and then act as competitor to endogenous RNA. Circular RNAs have promising prospect as clinical diagnostic markers or therapeutic molecules for the prophylaxis and treatment of diseases. Till today few reports have been published on circular RNAs due to low expression level. Originally these molecules were considered as by-products of alternative splicing and were named as a genetic accident or experimental errors (Liu L, Wang J, Khanabdali R et al (2017) Circular RNAs: isolation, characterization and their potential role in diseases. RNA Biol 14 (12): 1715-1721).

The broad occurrence of circular RNAs in vivo and the study of their structural and functional properties have caused demand for methods that allow efficient preparation of circular RNAs in vitro. Several methods have been reported to be useful in in vitro production of circular RNAs, including chemical and enzymatic methods, as well as the ones using modified self-splicing introns (group I and group II) with permuted introns and exons (PIE) strategies.

These engineered circular RNAs can carry an IRES (internal ribosome entry site) to effectively direct the expression of any downstream coding sequences in vivo, such as those for luciferase and the RBD of SARS-COV-2 spike protein (R. A. Wesselhoeft, P. S. Kowalski, D. G. Anderson, (2018) Engineering circular RNA for potent and stable translation in eukaryotic cells. Nat Commun 9, 2629; and Qu L. et al (2022) Circular RNA Vaccines against SARS-COV-2 and Emerging Variants. Cell 185 (10): 1728-1744.) As such, circular RNA is becoming an alternative to mRNA as therapeutic agents and vaccines.

Circular RNA has several advantages over linear mRNA as therapeutic agents or vaccines. It is more stable in vivo, and can achieve needed expression level with a lower dose. It is of low immunogenicity, and poses less toxicity or less adverse effects in treatment. For in vitro production, it does not require an expensive capping step or tailing step, which are usually required in producing mRNA products.

Needs remain for methods that allow efficient and economic preparation of circular RNAs in vitro, especially at industrial scales, to facilitate the research as well as applications in disease diagnosis and treatment.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “a method” means one method or more than one method.

The present disclosure provides novel methods of producing circular RNAs in vitro, as well as novel DNA constructs or RNA transcripts that allow the production of the circular RNAs via said methods. The present disclosure also provides compositions and kits that comprise the DNA constructs or RNA transcripts capable of producing circular RNAs, or the circular RNAs that produced via said methods.

In one aspect, the present disclosure provides a DNA construct for making a circular RNA, the DNA construct comprising an RNA polymerase promoter operably linked to a sequence coding for an RNA transcript capable of producing a circular RNA, said RNA transcript comprises the following elements operably linked to each other and arranged in the following sequence:
a) a first circularization element,
b) a target sequence, and
c) a second circularization element,
wherein the RNA transcript is capable of forming the circular RNA comprising the target sequence; and
wherein the RNA transcript further comprises at least one purification fragment that is absent in the circular RNA.

In certain embodiments, the RNA polymerase promoter is an RNA polymerase promotor derived from T7 virus, T6 virus, SP6 virus, T3 virus, or T4 virus.

In one aspect, the present disclosure provides an in vitro transcribed RNA transcript, said RNA transcript comprises the following elements operably linked to each other and arranged in the following sequence:
a) a first circularization element,
b) a target sequence, and
c) a second circularization element,
wherein the RNA transcript is capable of forming a circular RNA comprising the target sequence; and
wherein the RNA transcript further comprises at least one purification fragment that is absent in the circular RNA.

In certain embodiments, the at least one purification fragment is associated with the first circularization element, or associated with the second circularization element.

In certain embodiments, the RNA transcript has two purification fragments that are associated respectively with both the first circularization element and the second circularization element.

In certain embodiments, the at least one purification fragment is: a) located upstream of the first circularization element (i.e. 5′ purification fragment), b) located downstream of the second circularization element (i.e. 3′ purification fragment), c) or any combination of a) and b).

In certain embodiments, the at least one purification fragment is: a) attached to the 5′ end of the first circularization element (i.e. 5′ purification fragment), or b) attached to the 3′ end of the second circularization element (i.e. 3′ purification fragment), or any combination of a) and b).

In certain embodiments, the RNA transcript provided herein has both the 5′ purification fragment and the 3′ purification fragment, and wherein the 5′ purification fragment and the 3′ purification fragment are either identical or different in nucleotide sequence.

In certain embodiments, each of the at least one purification fragment has no more than 90% (or no more than 80%, no more than 70%, no more than 60%, no more than 50%) sequence identity to the circular RNA in a given length of 15-20 nucleotides.

In certain embodiments, the purification fragment comprises a poly(A) tract, poly(T) tract, poly(U) tract, poly(C) tract, poly(G) tract, poly(AC) tract, poly(AG) tract, poly(CT) tract, poly(CU) tract, poly(AT) tract, or poly(AU) tract. In certain embodiments, the purification fragment comprises a poly(A) tract, poly(U) tract, poly(C) tract, poly(G) tract, poly(AC) tract, poly(AG) tract, poly(CU) tract, or poly(AU) tract.

In certain embodiments, the purification fragment (e.g. the poly(A) tract) has a length ranging from 15 to 200 nucleotides, optionally from 15 to 150 nucleotides.

In certain embodiments, the first circularization element and the second circularization element can be derived from a self-splicing system.

In certain embodiments, the first circularization element comprises a 3′ portion of a self-splicing element comprising a 3′ splice site, and the second circularization element comprises a 5′ portion of the self-splicing element comprising a 5′ splice site.

In certain embodiments, the self-splicing element is a Group I intron, a Group II intron, or a hairpin ribozyme.

In certain embodiments, the Group I intron is derived from phage T4 thymidylate synthase (td) gene, Cyanobacterium Anabaena sp. pre-tRNA-Leu gene or Tetrahymena gene.

In certain embodiments, the Group II intron is derived from L. lactis Ll.LtrB gene, or derived from yeast.

In certain embodiments, the hairpin ribozyme is derived from bacteria, eukaryotes, or plant virus RNA satellites (e.g., tobacco ringspot virus (TRSV) satellite RNA, chicory yellow mottle virus (sCYMV) satellite RNA, or arabis mosaic virus (sARMV) satellite RNA).

In certain embodiments, the target sequence comprises a target protein coding region.

In certain embodiments, the target protein is a therapeutic protein (e.g. an antibody, cytokine, peptide hormone, etc.), a prophylactic protein (e.g. a protein vaccine, etc.), a nuclease (e.g. Cas protein, recombinase, etc.), or a receptor (e.g. chimeric antigen receptor, etc.).

In certain embodiments, the target sequence further comprises an internal ribosome entry site (IRES) operably linked to the target protein coding region. In certain embodiments, the IRES is operably linked upstream or downstream of the target protein coding region.

In certain embodiments, the IRES is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eaF4G, Coxsackievirus B3 (CVB3) or Coxsackievirus A (CVB1/2).

In certain embodiments, the target sequence further comprises a 5′ Untranslated Region (UTR) and/or a 3′ UTR operably linked to the target protein coding region.

In certain embodiments, the target sequence comprises a biologically active RNA or a precursor of the biologically active RNA.

In certain embodiments, the biologically active RNA comprises a short hairpin RNA, transfer RNA (tRNA), short interfering RNA, microRNA, or guide RNA.

In certain embodiments, the RNA transcript further comprises at least one homology arm. In certain embodiments, the homology arm is located upstream (i.e. 5′ homology arm) of the first circularization element and/or downstream (i.e. 3′ homology arm) of the second circularization element. In certain embodiments, the homology arm is located upstream (i.e. 5′ homology arm) of the 3′ portion of the self-splicing element and/or downstream (i.e. 3′ homology arm) of the 5′ portion of the self-splicing element.

In certain embodiments, wherein the homology arm comprises at least one ALU element derived from ALU repeats.

In certain embodiments, the homology arm is: a) inserted between the 5′ purification fragment and the first circularization element and/or inserted between the 3′ purification fragment and the second circularization element; or b) located upstream of the 5′ purification fragment and/or downstream of the 3′ purification fragment.

In certain embodiments, the RNA transcript further comprises at least one spacer, optionally the spacer is located between the first circularization element (e.g. the 3′ portion of the self-splicing element) and the target sequence, and/or between the target sequence and the second circularization element (e.g. the 5′ portion of the self-splicing element).

In certain embodiments, the RNA transcript further comprises at least one spacer. In certain embodiments, the spacer is located between the first circularization element (e.g. the 3′ portion of the self-splicing element) and the target sequence, and/or between the target sequence and the second circularization element (e.g. the 5′ portion of the self-splicing element). In certain embodiments, the spacer is located between the first circularization element (e.g. the 3′ portion of the self-splicing element) and the IRES sequence, and/or between the target sequence and the second circularization element (e.g. the 5′ portion of the self-splicing element). In certain embodiments, the spacer is located between the first circularization element (e.g. the 3′ portion of the self-splicing element) and the target protein coding region, and/or between the target protein coding region and the second circularization element (e.g. the 5′ portion of the self-splicing element). In certain embodiments, the target sequence comprises two complementary spacer sequences flanking the target sequence.

In one aspect, the present disclosure provides a DNA construct comprising the following elements operably linked to each other and arranged in 5′ to 3′ sequence:
a) a first purification fragment, optionally a first poly(A) tract, and
b) a multi-cloning site comprising or consisting essentially of one or more cloning sites for inserting a target sequence.

In certain embodiments, the DNA construct further comprises a second purification fragment downstream of the multi-cloning site.

In certain embodiments, the DNA construct further comprises a first circularization element between the first purification fragment and the multi-cloning site, and/or a second circularization element downstream of the multi-cloning site. In certain embodiments, the second circularization element is between the multi-cloning site and the second purification fragment.

In certain embodiments, the DNA construct further comprises an RNA polymerase promoter upstream of the first purification fragment.

In one aspect, the present disclosure provides a DNA construct comprising the following elements operably linked to each other and arranged in the following sequence:
a) an RNA polymerase promoter,
b) a first circularization element,
c) a multi-cloning site, and
d) a second circularization element,
wherein the DNA construct further comprises at least one purification fragment that is downstream of the RNA polymerase promoter.

In certain embodiments, one of the purification fragments is located upstream of the first circularization element, and/or downstream of the second circularization element.

In one aspect, the present disclosure provides a method of producing the RNA transcript provided herein, the method comprising transcribing from the DNA construct provided herein, thereby obtaining the RNA transcript provided herein.

In one aspect, the present disclosure provides a method of producing the RNA transcript provided herein, the method comprising:
a) providing a precursor RNA which differs from the RNA transcript provided herein in lacking the purification fragment, and
b) adding the purification fragment to the precursor RNA, thereby obtaining the RNA transcript provided herein.

In certain embodiments, the precursor RNA differs from the RNA transcript provided herein only in lacking the purification fragment.

In certain embodiments, the purification fragment is added to: a) a position located upstream of the first circularization element, b) a position located downstream of the second circularization element, c) or any combination of a) and b).

In certain embodiments, the purification fragment is: a) attached to the 5′ end of the first circularization element, or b) attached to the 3′ end of the second circularization element, c) or any combination of a) and b).

In one aspect, the present disclosure provides a method of producing a circular RNA, the method comprising:
a) providing the RNA transcript provided herein, and
b) allowing self-circularization of the RNA transcript to form the circular RNA.

In certain embodiments, the RNA transcript in step a) has been enriched using a capturing agent that specifically binds to the purification fragment present in the RNA transcript.

In certain embodiments, the method further comprises purifying the circular RNA from the product obtained in step b) by a capturing agent, wherein the capturing agent specifically binds to the purification fragment in the RNA transcript and optionally in a by-product but absent in the circular RNA.

In certain embodiments, the capturing agent comprises a nucleic acid fragment hybridizable to the purification fragment under stringent conditions. In certain embodiments, the capturing agent comprises a nucleic acid fragment at least 80%, 85%, 90%, 95%, or 100% complementary to the nucleotide sequence of the purification fragment. In certain embodiments, the capturing agent comprises a protein, a peptide, a polysaccharide, or a small molecule that is able to bind to the purification fragment as provided herein.

In certain embodiments, the capturing agent is immobilized.

In certain embodiments, the RNA transcript in step b) undergoes self-circularization either in solution or on solid matrix where the capturing agent is immobilized.

In certain embodiments, the purification fragment comprises a poly(A) tract and the capturing agent comprises deoxythymidine oligonucleotide (oligo(dT)).

In certain embodiments, the RNA transcript in step a) is transcribed from the DNA construct provided herein.

In certain embodiments, the method further comprises, before step a), transcribing from the DNA construct provided herein to produce the RNA transcript provided herein.

In certain embodiments, the RNA transcript in step a) is obtained by the method provided herein.

In one aspect, the present disclosure provides a composition of circular RNA produced using the method provided herein.

In one aspect, the present disclosure provides a composition comprising the DNA construct provided herein, or the RNA transcript provided herein.

In one aspect, the present disclosure provides a kit comprising the DNA construct provided herein.

In one aspect, the present disclosure provides a kit for producing the RNA transcript provided herein, comprising a reagent useful for adding the purification fragment to the precursor of the RNA transcript.

In certain embodiments, the kit further comprises a capturing agent that specifically binds to the purification fragment that is present in the RNA transcript but absent in the circular RNA.

In certain embodiments, the purification fragment comprises a poly(A) tract.

In certain embodiments, the capturing agent comprises oligo(dT).

### BRIEF DESCRIPTION OF FIGURES

FIG. 1 shows schematic drawing for an illustrative DNA construct and an illustrative process showing the production of a circular RNA from the illustrative DNA construct. The DNA construct as shown is a circular plasmid DNA which comprises a plasmid backbone, a circular RNA region, a site for RNA polymerase promoter (such as T7 as shown), one or two purification fragment(s) (PF), and a cut site for restriction enzymes (ideally, close to or at the 3′ end of PF as shown). The process of circular RNA production comprises linearization of plasmid DNA, in vitro transcription to generate the RNA precursor, circularization to form the circular RNA, and purification by removing all RNA fragments carrying PFs by an affinity matrix.

FIG. 2 shows four categories of exemplified RNA precursors based on different arrangements of elements and the positioning of purification fragment. The first circularization element used herein is the 3′ portion of the self-splicing element, designated as 3′ intron, which comprises portions of 3′ intron and Exon2 of a conventional PIE strategy. The second circularization element is the 5′ portion of the self-splicing element, designated as 5′ intron, which comprises portions of 5′ intron and Exon1 of a conventional PIE strategy. The purification fragment is designated as PF. The RNA transcripts having no purification fragments are designated as “Ctrl”. Category I shows RNA precursors comprising two circularization elements, and one or two purification fragments located at 5′ end of the first circularization element (designated as I-5 PF), at 3′ end of the second circularization element (designated as I-3 PF), or at both ends (designated as I-5 PF+3 PF). Category II shows RNA precursors comprising two circularization elements, two spacers, and one or two purification fragments located at 5′ end of the first circularization element (designated as II-5 PF), at 3′ end of the second circularization element (designated as II-3 PF), or at both ends (designated as II-5 PF+3 PF). Category III shows RNA precursors comprising two circularization elements, two spacers, two homology arms, and one or two purification fragments located at 5′ end of the 5′ homology arm (designated as III-5 PF), at 3′ end of the 3′ homology arm (designated as III-3 PF), or at both ends (designated as III-5 PF+3 PF). Category IV shows RNA precursors comprising two circularization elements, two spacers, two homology arms, and one or two purification fragments located between the 5′ homology arm and the first circularization element (designated as IV-5 PF), between the second circularization element and the 3′ homology arm (designated as IV-3 PF), or at both positions (designated as IV-5 PF+3 PF).

FIG. 3A shows linear RNA precursors transcribed from the DNA constructs as provided herein. Here the purification fragment is a poly-A tract and the elements are arranged as in Category III in FIG. 2. Ctrl: the RNA transcript not comprising any purification fragment as a negative control. 5A: the RNA transcript comprising a poly-A tract at the 5′ end of the RNA transcript. 3A: the RNA transcript comprising a poly-A tract at the 3′ end of the RNA transcript. 53A: the RNA transcript comprising two poly-A tracts each positioned at one end of the RNA transcript. The “3′ intron” as denoted represents the first circularization element, comprising the sequences of 3′ intron and Exon2 of a conventional PIE strategy. The “5′ intron” as denoted represents the second circularization element, comprising the sequences of 5′ intron and Exon1 of a conventional PIE strategy.

FIG. 3B shows an experimental workflow for the production, purification, and characterization of circular RNAs.

FIG. 4 shows an analysis of RNA products on agarose gels. Plasmid DNAs of four constructs as described in FIG. 3A were prepared and linearized. After in vitro transcription and circularization, the RNA products were loaded to Oligo-dT magnetic beads or columns containing Oligo-dT resins. Fractions were collected at sequential steps and analyzed on agarose gels and by capillary electrophoresis (FIG. 5). Input: the RNA product after in vitro transcription and circularization, which contains both circular RNAs and poly-A-containing linear RNA precursors, as well as all the byproducts. FT: the flow-through fraction, which contains circular RNAs. E: the eluted fraction, which contains the unprocessed poly-A-containing linear RNA precursors and the processed poly-A-containing RNA fragments. W: the fraction from a washing step between binding and elution that was used to wash off nonspecifically bound RNA products. P: indicates where the RNA precursors migrate on the agarose gel. R: indicates where the circular RNA products migrate on the agarose gel.

FIGS. 5A-5E show an analysis of RNA products by capillary electrophoresis. RNA products as described in FIG. 4 were also analyzed by capillary electrophoresis (Agilent 5200 fragment analyzer): Ctrl (FIG. 5A), 5A (FIG. 5B), 3A (FIG. 5C) and 53A (FIG. 5D). “Input” stands for the RNA product after in-vitro transcription and circularization, which was loaded for Oligo-dT purification. “FT” (flow-through) stands for the flow-through fraction. The circularization efficiency and purity of circRNA were calculated and summarized in FIG. 5E.

FIG. 6 shows the transfection results of the purified GFP circRNA into HEK 293T cells. The Control sample and the eGFP circRNA sample were separately transfected by lipofectamine reagents. Images were taken on a fluorescence microscope under bright field and under a GFP setting.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

The present disclosure provides novel composition and methods useful for preparation of circular RNA. In general, in vitro production of circular RNA goes through two steps, one is in vitro transcription to produce a linear RNA precursor, and the other is the circularization step to form the circular RNA from precursors. The circularization is normally not 100% efficient, leaving certain amount of precursor RNA and intermediate needs to be removed from circularized products. Due to the closeness of the precursor and circular RNA in size and physical properties, their full separation is difficult to achieve with conventional chromatographic methods like ion exchange or hydrophobic interaction. Current purification processes for circular RNA known in the art uses an HPLC SEC column, which is not scalable to an industrial scale, and is expensive. The present disclosure discloses a series of designed DNA constructs and RNA transcripts that contain one or more purification fragments (PF). These purification fragments are not present in the circular RNA after circularization, and are found to be surprisingly effective in facilitating removal of linear RNA transcripts and circularization intermediates, thereby achieving purification of circular RNA products in a convenient and scalable manner. The purification step can be carried out in common chromatographic equipment used in laboratories as well as in manufacturing sites. A desired purity (e.g. ≥80%) can be easily achieved. This is proven to be especially desirable for manufacturing of therapeutic agents and vaccines which need robust and scalable purification processes.

### DNA Constructs and RNA Transcripts for Circular RNA Preparation

The present disclosure provides a DNA construct for making a circular RNA, the DNA construct comprising an RNA polymerase promoter operably linked to a sequence coding for an RNA transcript capable of producing a circular RNA, said RNA transcript comprises the following elements operably linked to each other and arranged in the following sequence:
a) a first circularization element,
b) a target sequence, and
c) a second circularization element,
wherein the RNA transcript is capable of forming the circular RNA comprising the target sequence; and wherein the RNA transcript further comprises at least one purification fragment that is absent in the circular RNA.

The present disclosure also provides an in vitro transcribed RNA transcript, said RNA transcript comprises the following elements operably linked to each other and arranged in the following sequence:
a) a first circularization element,
b) a target sequence, and
c) a second circularization element,
wherein the RNA transcript is capable of forming a circular RNA comprising the target sequence; and wherein the RNA transcript further comprises at least one purification fragment that is absent in the circular RNA.

As used herein, the terms “circRNA” or “circular RNA” are used interchangeably and refers to a polyribonucleotide that forms a circular structure through covalent bonds.

As used herein, the term “DNA construct” refers to a piece of DNA into which a foreign DNA fragment can be or has been inserted. A DNA construct is of any length and of any sequence and can be either linear or circular. A DNA construct can be synthesized (e.g., using PCR), or can be derived from a virus, plasmid, or cell of a higher organism. The term includes linear DNA fragments (e.g., PCR products, linearized plasmid fragments), plasmid vectors, viral vectors, cosmids, bacterial artificial chromosomes (BACs), yeast artificial chromosomes (YACs), and the like. A DNA construct can comprise, for example, an origin of replication, a selectable marker or reporter gene, such as antibiotic resistance or GFP, an RNA polymerase promoter and/or an RNA polymerase terminator. A DNA construct can be PCR-amplified linear fragment, which contains essential elements for making the RNA transcript provided herein by in vitro transcription.

The term “nucleic acid” or “nucleotide” include naturally-occurring species as well as any analogs, variants, and any mimetics thereof that are capable of hybridizing to a naturally-occurring nucleic acid in a sequence-specific manner, for example, capable of hybridizing to two nucleic acids such that ligation can occur between the two hybridized nucleic acids, or are capable of being used as a template for replication of a particular nucleotide sequence. Analogs, variants, and any mimetics of a naturally-occurring nucleotide can have modifications in the chemical structure of the base, sugar and/or phosphate. Examples of modifications in base include, but are not limited to, 5′-position pyrimidine modifications, 8′-position purine modifications, modifications at cytosine exocyclic amines, and substitution of 5-bromo-uracil. Examples of nucleotide analogs include 5-methoxyuridine, pseudouridine, 1-methylpseudouridine, and 6-methyladenosine. Examples of modifications in sugar include but are not limited to, sugar-modified ribonucleotides in which the 2′-OH is replaced by a group such as an H, alkoxy group, alkyl group, halo, SH, SR, NH2, NHR, NR2, or CN, wherein R is an alkyl moiety. Examples of modifications in phosphodiester linkages include without limitation, methylphosphonate, phosphorothioate and peptide linkages. Nucleotide are also meant to include nucleotides with bases such as inosine, queuosine, xanthine; sugars such as 2′-methyl ribose. In certain embodiments, a nucleic acid molecule can be DNA or RNA.

The term “RNA” or “ribonucleic acid” include native RNA species having bases selected from the group consisting of adenine (A), uracil (U), cytosine (C), or guanine (G), as well as any non-native RNA, or analogs, variants, and any mimetics thereof, that are capable of base pairing with a native RNA.

The term “DNA” or “deoxyribonucleic acid” include native DNA species having bases selected from the group consisting of adenine (A), thymine (T) cytosine (C), or guanine (G), as well as any non-native DNA, or analogs, variants, and any mimetics thereof, that are capable of base pairing with a native DNA.

“Percent (%) sequence identity” with respect to nucleotide sequence (or amino acid sequence) is defined as the percentage of nucleotide (or amino acid) residues in a candidate sequence that are identical to the nucleotide (or amino acid) residues in a reference sequence, after aligning the sequences and, if necessary, introducing gaps, to achieve the maximum correspondence. Alignment for purposes of determining percent nucleotide (or amino acid) sequence identity can be achieved, for example, using publicly available tools such as BLASTN, BLASTP (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23 (21): 2947-8 (2007)), and ALIGN or MegAlign (DNASTAR) software. Those skilled in the art may use the default parameters provided by the tool, or may customize the parameters as appropriate for the alignment, such as for example, by selecting a suitable algorithm.

The term “complementary” or “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds with another nucleic acid sequence (e.g., 5, 6, 7, 8, 9, 10 out of 10 being 50%, 60%, 70%, 80%, 90%, and 100% complementary). Percent complementarity of can be determined routinely using basic local alignment search tools (BLAST programs) (Altschul (1990) J. Mol. Biol. 215, 403-410; Zhang and Madden (1997) Genome Res. 7, 649-656).

The term “hybridizing” or “hybridize” refer to the pairing of substantially complementary or complementary nucleic acid sequences within two different molecules or within two fragments. Pairing can be achieved by any process in which a nucleic acid sequence binds to a substantially or fully complementary sequence through base pairing to form a hybridization complex.

“Stringent condition” as used herein refers to a condition under which a nucleotide sequence will hybridize to its target nucleotide sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the melting temperature (Tm) for the specific sequence at a defined ionic strength and pH. Thus, oligonucleotides are chosen that are sufficiently complementary to the target, i.e., that hybridize sufficiently well and with sufficient specificity, to give the desired effect, while striving to avoid significant off-target effects, i.e. not to directly bind to other than the intended target.

As used herein, the term “downstream” with respect to nucleotide sequence, means toward the 3′ end of the nucleotide sequence.

As used herein, the term “upstream” with respect to nucleotide sequence, means toward the 5′ end of the nucleotide sequence.

As used herein, the term “operably linked” refers to an arrangement of elements wherein the components so described are configured so as to perform their usual function. Thus, for example, when a given promoter is operably linked to a coding DNA sequence, the promoter is capable of effecting the expression of the coding DNA sequence in the presence of proper enzymes such as RNA polymerase. Expression is meant to include the transcription of any one or more of transcription of a circular RNA, recombinant nucleic acid encoding a circular RNA, or mRNA from a DNA or RNA template and can further include translation of a protein from an mRNA template or a circular RNA comprising an IRES sequence. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “RNA polymerase promoter” refers to a DNA regulatory region capable of recruiting RNA polymerase (e.g. RNA polymerase II) and initiating transcription of DNA into RNA. An RNA polymerase promoter can have a transcription initiation site at its 3′ terminus, from which the transcription proceeds. The promoter sequence includes the minimum number of bases with elements necessary to initiate transcription at levels detectable above background. Normally the RNA polymerase promoter sequence has a protein binding region responsible for the binding of RNA polymerase. In certain embodiments, the RNA polymerase promoter can be selected from, but is not limited to, an RNA polymerase promoter derived from T7 virus, T6 virus, SP6 virus, T3 virus, or T4 virus.

In certain embodiments, the DNA construct further comprises an RNA polymerase terminator. As used herein, “RNA polymerase terminator” refers to the element comprises at least one discrete terminator sequence, where the transcription by RNA polymerase terminates. Terminators cause release of the RNA and dissociation of the transcription complex. In certain embodiments, the RNA polymerase terminator can be selected from, but not limited to, an RNA polymerase terminator derived from T7 virus, T6 virus, SP6 virus, T3 virus, or T4 virus.

The “RNA transcript” used herein refers to a linear nucleic acid fragment that is transcribed from a given DNA construct. In one embodiment, the sequence encoding for an RNA transcript is comprised in a DNA construct and located at the downstream of an RNA polymerase promoter. For example, the RNA transcript provided herein can be transcribed from the DNA construct provided herein. In another embodiment, the RNA transcript is provided or synthesized in vitro.

The RNA transcript provided herein is capable of producing a circular RNA. The RNA transcript provided herein comprises the following elements operably linked to each other and arranged in the following sequence (e.g. from 5′ to 3′): a) a first circularization element, b) a target sequence, and c) a second circularization element, and further comprises at least one purification fragment that is absent in the circular RNA, and wherein the RNA transcript is capable of forming the circular RNA comprising the target sequence.

### 1. Circularization Element

As used herein, the term “circularization element” refers to at least one nucleic acid fragment that is operably linked to a linear RNA, in such a way that facilitates self-circularization of the linear RNA to form a circular RNA. The circular RNA produced from the RNA transcript provided herein comprises the target sequence. In certain embodiments, the circularization element in the RNA transcript comprises or is an RNA fragment.

In certain embodiments, the first circularization element is located upstream of the target sequence, and/or the second circularization element is located downstream of the target sequence. In certain embodiments, the RNA transcript comprises at least one pair of the circularization elements, located upstream and downstream, respectively, of the target sequence.

In certain embodiments, the circularization elements are derived from a self-splicing system. In certain embodiments, the circularization elements can be derived from Group I intron self-splicing system, Group II intron self-splicing system or a hairpin ribozyme.

The Group I intron self-splicing system involves group I introns that are mainly located within genomic ribosomal RNA regions of eukaryotic microorganisms. Group I introns are characterized by a linear array of conserved sequences and structural features, and are excised by two successive transesterifications. Group I introns recruit an external guanosine as a nucleophile to initiate splicing. During the process, the 3′ hydroxyl group of a guanosine nucleotide engages in a transesterification reaction at the 5′ exon-intron junction (5′ splice site), which results in excision of the 5′ intron portion, leaving an intermediate having a free 3′-hydroxyl group at the end of the 5′ exon. The freed hydroxyl group engages in a second transesterification at the 3′ exon-intron junction (3′ splice site), resulting in circularization of the intervening region and excision of the 3′ intron portion. See, for details, Wesselhoeft R. A. et al., Engineering circular RNA for potent and stable translation in eukaryotic cells, Nature Communications, (2018) 9:2629.

Group II intron self-splicing system are mobile genetic elements that autocatalytically splice themselves from precursor RNAs by using a transesterification reaction similar to the classical spliceosomal splicing reaction. Group II introns have been found in bacteria and in the mitochondrial (mt) and chloroplast (cp) genomes of fungi, plants, protists, and an annelid worm. Group II intron RNAs are characterized by a conserved secondary structure, which spans 400-800 nts and is organized into six domains, DI-VI, radiating from a central “wheel”. These domains interact to form a conserved tertiary structure that brings together distant sequences to form an active site. The active site binds the splice sites and branch-point nucleotide residue and uses specifically bound Mg++ ions to activate the appropriate bonds for catalysis.

Group II introns catalyze their own splicing via two sequential transesterification reactions, generating a branched lariat intermediate and a lariat-intron. Two intron portions having complementary sequences can hybridize, thereby juxtaposing the branch point of the 5′-intron and the 3′-intron-exon junction (3′-splice site) for nucleophilic attack and cleavage. After release of the 3′ exon, the terminal 2′-OH group of the 3′-splice site attacks the 5′-intron-exon junction (5′-splice site), joining the two exons and releasing the intron lariat (circularized) RNA with a 2′ to 5′ phosphodiester bond. See, for details, Llorente-Cortés, V. et al, Advances in Experimental Medicine and Biology, Volume 1087, Chapter in Circular RNAs: Biogenesis and Functions, published by Springer; Petkovic, S. et al, RNA circularization strategies in vivo and in vitro, Nucleic Acids Research, 2015, Vol. 43, No. 4, 2454-2465; Lambowitz, A. M. et al., Cold Spring Harb Perspect Biol. 2011 August; 3 (8): a003616.

Hairpin ribozyme (HPR) is another system that permits self-splicing and generation of circular RNA. A linear RNA with HPR can fold into two cleavage-active conformations, both addressing one of the two possible cleavage sites. After first cleavage took place in either conformation, an RNA fragment is cleaved, generating a 5′-OH and 2′,3′-cyclic phosphate (2′,3′-cP). The hairpin ribozyme is then refolded into the alternative conformation, which allows the second cleavage reaction to occur, generating another cleaved RNA fragment as well as a 5′-OH and 2′,3′-cP. As a result, the cleaved intermediate will contain a 5′-OH (at the 3′ exon) and a 2′,3′-cyclic phosphate (at the 5′ exon), which can be ligated to produce the target circRNA. See, for details, Chen, X J et al, Circular RNA: Biosynthesis in vitro, Front. Bioeng. Biotechnol., 30 Nov. 2021; Hieronymus, R. et al, RNA self-splicing by engineered hairpin ribozyme variants, Nucleic Acids Research, Volume 50, Issue 1, 11 Jan. 2022, Pages 368-377, Diegelman, A. M. et al., Generation of circular RNAs and trans-cleaving catalytic RNAs by rolling transcription of circular DNA oligonucleotides encoding hairpin ribozymes, Nucleic Acids Research, 1998, Vol. 26, No. 13:3235-3241. Hairpin ribozymes are well known in the art, and numerous natural hairpin ribozymes as well as artificially designed hairpin ribozymes are also known (Christina E Weinberg et al., Identification of over 200-fold more hairpin ribozymes than previously known in diverse circular RNAs, Nucleic Acids Res. 2021 Jun. 21; 49 (11): 6375-6388). In certain embodiments, the hairpin ribozyme is derived from bacteria, eukaryotes, or plant virus RNA satellites (e.g., tobacco ringspot virus (TRSV) satellite RNA, chicory yellow mottle virus (sCYMV) satellite RNA, or arabis mosaic virus (sARMV) satellite RNA).

In certain embodiments, the first circularization element comprises a 3′ portion of a self-splicing element comprising a 3′ splice site, and the second circularization element comprises a 5′ portion of the self-splicing element comprising a 5′ splice site.

As used herein, the term “splice site” refers to a dinucleotide that is partially or fully included in a self-splicing element and between which a phosphodiester bond is cleaved during RNA circularization.

As used herein, a “3′ portion of a self-splicing element” is a contiguous sequence that has at least 75% (e.g., at least 80%, at least 85%, at least 90%, at least 95%, 100%) sequence identity to a 3′ proximal fragment of a natural group I intron, a natural group II intron, or a natural hairpin ribozyme, including the 3′ splice site dinucleotide, and optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length).

As used herein, a “5′ portion of a self-splicing element” is a contiguous sequence that has at least 75% (e.g., at least 80%, at least 85%, at least 90%, at least 95%, 100%) sequence identity to a 5′ proximal fragment of a natural group I intron, a natural group II intron, or a natural hairpin ribozyme, including the 5′ splice site dinucleotide and, optionally, the adjacent exon sequence at least 1 nucleotide in length (e.g., at least 5 nucleotides in length, at least 10 nucleotides in length, at least 15 nucleotides in length, at least 20 nucleotides in length, at least 25 nucleotides in length, at least 50 nucleotides in length).

In certain embodiments, the Group I intron is derived from phage T4 thymidylate synthase (td) gene, Cyanobacterium Anabaena sp. pre-tRNA-Leu gene or Tetrahymena gene. In certain embodiments, the Group II intron is derived from L. lactis Ll.LtrB gene, or is derived from yeast.

In certain embodiments, the 3′ portion of the self-splicing element as provided herein is encoded by the nucleic acid sequence comprising SEQ ID NO: 7 (AACAATAGATGACTTACAACTAATCGGAAGGTGCAGAGACTCGACGGGAGCTA CCCTAACGTCAAGACGAGGGTAAAGAGAGAGTCCAATTCTCAAAGCCAATAGGC AGTAGCGAAAGCTGCAAGAGAATGAAAATCCGTTGACCTTAAACGGTCGTGTGG GTTCAAGTCCCTCCACCCCCA).

In certain embodiments, the 5′ portion of the self-splicing element as provided herein is encoded by the nucleic acid sequence comprising SEQ ID NO: 12 (AGACGCTACGGACTTAAATAATTGAGCCTTAAAGAAGAAATTCTTTAAGTGGAT GCTCTCAAACTCAGGGAAACCTAAATCTAGTTATAGACAAGGCAATCCTGAGCC AAGCCGAAGTAGTAATTAGTAAG).

### 2. Target Sequence

As used herein, the term “target sequence” can be any sequence of interest that could be inserted into the sequence coding for an RNA transcript and comprised in the circular RNA transcribed from the RNA transcript provided herein. The target sequence comprises a protein coding or noncoding region. In certain embodiments, the target sequence comprises a target protein coding region. In certain embodiments, the target sequence comprises a biologically active RNA or a precursor of the biologically active RNA. In certain embodiments, the target sequence comprises or consists essentially of one or more cloning sites.

The target sequence can comprise a target protein coding region. In certain embodiments, the protein coding region encodes a protein of eukaryotic or prokaryotic origin. In certain embodiments, the protein coding region encodes human protein or non-human protein. In certain embodiments, the protein can be any protein for research use, therapeutic use or diagnostic use. In some embodiments, the protein can be a therapeutic protein, for example, an antibody, cytokine, peptide hormone, etc. In certain embodiments, the protein can be a prophylactic protein, for example, a protein vaccine, etc. In certain embodiments, the protein can be a nuclease, for example, a Cas protein, recombinase, etc. In certain embodiments, the protein can be a receptor, for example, a chimeric antigen receptor, etc. In some embodiments, the protein can be selected from, but not limited to, hFIX, SP-B, VEGF-A, human methylmalonyl-CoA mutase (hMUT), CFTR, cancer self-antigens, and additional gene editing enzymes like Cpf1, zinc finger nucleases (ZFNs) and transcription activator-like effector nucleases (TALENs).

In certain embodiments, the target sequence further comprises a 5′ Untranslated Region (UTR) and/or a 3′ UTR operably linked to the target protein coding region. The 5′ UTR can be selected from, but not limited to, a 5′ UTR of human beta globin, Xenopus laevis beta globin, human alpha globin, Xenopus laevis alpha globin, rubella virus, tobacco mosaic virus, mouse Gtx, dengue virus, heat shock protein 70 kDa protein 1A, tobacco alcohol dehydrogenase, tobacco etch virus, turnip crinkle virus, or the adenovirus tripartite leader. The 3′ UTR can be selected from, but not limited to, a 3′ UTR of human beta globin, human alpha globin Xenopus beta globin, Xenopus alpha globin, human prolactin, human GAP-43, human eEF1a1, human Tau, human TNF alpha, dengue virus, hantavirus small mRNA, bunyanavirus small mRNA, turnip yellow mosaic virus, hepatitis C virus, rubella virus, tobacco mosaic virus, human IL-8, human actin, human GAPDH, human tubulin, hibiscus chlorotic rinsgpot virus, woodchuck hepatitis virus post translationally regulated element, sindbis virus, turnip crinkle virus, tobacco etch virus, or Venezuelan equine encephalitis virus. Wild-type 5′ UTR and/or 3′ UTR sequences can also be modified and be effective in the invention.

In certain embodiments, the target sequence further comprises an internal ribosome entry site (IRES) operably linked to the target protein coding region. In certain embodiments, the IRES is operably linked upstream or downstream of the target protein coding region. The IRES sequence can be selected from, but not limited to, an IRES sequence of a Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua a picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eaF4G, Coxsackievirus B3 (CVB3) or Coxsackievirus A (CVB1/2). Wild-type IRES sequences can also be modified and be effective in the invention. In some embodiments, the IRES sequence is about 50 nucleotides in length.

In certain embodiments, the target sequence can comprise a non-coding RNA. In certain embodiments, the target sequence can comprise a biologically active RNA or a precursor of the biologically active RNA. In certain embodiments, the biologically active RNA comprises a short hairpin RNA, transfer RNA (tRNA), short interfering RNA, microRNA, or guide RNA. The precursor of the biologically active RNA can be processed to give rise to the biologically active RNA. For example, a short hairpin RNA, which can be processed into a biologically active siRNA, can be a precursor of the siRNA.

### 3. Purification Fragment

In certain embodiments, the RNA transcript has at least one purification fragment.

As used herein, the term “purification fragment” refers to a nucleic acid fragment that is present in the RNA transcript but absent in the circular RNA produced from the RNA transcript. At least one purification fragment is located within the RNA transcript but not within the target sequence. No purification fragment is to be included in the circular RNA produced from the RNA transcript, and its presence does not significantly reduce production of the circular RNA. In other words, the RNA transcript comprising at least one purification fragment provided herein can still be circularized to produce the circular RNA. In certain embodiments, the purification fragment in the RNA transcript comprises or is an RNA fragment.

The nucleic acid sequence of the purification fragment can be sufficiently distinguishable from any equal length fragment of the circular RNA. Methods are known in the art to design unique nucleotide sequences, useful as the purification fragment, to distinguish from a given circular RNA sequence.

In certain embodiments, the purification fragment can be an RNA sequence that is present in the RNA transcript but absent in the circular RNA produced from the RNA transcript.

In certain embodiments, each of at least one of the purification fragments has no more than 90% (or no more than 80%, no more than 70%, no more than 60%, no more than 50%) sequence identity to the circular RNA in a given length of 15-20 nucleotides (nt), or 15nt-25nt, or 15nt-40nt, or 15nt-50nt.

For example, when the length of the purification fragment is shorter than or within the range of 15-20nt, 15nt-25nt, or 15nt-40nt, or 15nt-50nt, the purification fragment can have no more than 90%, no more than 80%, no more than 70%, no more than 60%, no more than 50%, or no more than 40% sequence identity to the circular RNA over the entire length of the purification fragment.

For another example, when the length of the purification fragment is longer than the upper limit of the given length range of 15-20nt, 15nt-25nt, or 15nt-40nt, or 15nt-50nt, then a given length fragment of the purification fragment can have no more than 90%, no more than 80%, no more than 70%, no more than 60%, no more than 50%, or no more than 40% sequence identity to an equal length fragment of the circular RNA.

In certain embodiments, the at least one purification fragment differs from an equal length fragment of the circular RNA by at least 1, 2, 3, 4, 5, 6, 7, 8, 9, 10, or even more nucleotides.

In certain embodiments, the at least one purification fragment has a length ranging from 15 to 500 nucleotides, from 15 to 300 nucleotides, from 15 to 200 nucleotides, from 15 to 150 nucleotides, from 15 to 100 nucleotides, from 15 to 50 nucleotides, from 15 to 25 nucleotides, from 20 to 500 nucleotides, from 20 to 300 nucleotides, from 20 to 200 nucleotides, from 20 to 150 nucleotides, from 20 to 100 nucleotides, from 20 to 50 nucleotides, or from 20 to 25 nucleotides.

In certain embodiments, the purification fragment is not part of the circularization element.

In certain embodiments, the purification fragment comprises or consists essentially of a homo-polymeric or homo-oligomeric sequence, such as a poly(A), poly(U), poly(T), poly(C), poly(G), poly(AT), poly(TC), poly(AU), poly(UC), poly(AC), poly(CA), poly(AG), poly(GA), to name just a few. In certain embodiments, the purification fragment comprises or consists essentially of a homo-polymeric or homo-oligomeric RNA sequence, such as a poly(A), poly(U), poly(C), poly(G), poly(AU), poly(UC), poly(AC), poly(CA), poly(AG), or poly(GA), to name just a few. The term “poly” as used herein with respect to purification fragment is intended to be any suitable length of nucleotides (i.e. more than 1).

In certain embodiments, the purification fragment comprises or consists essentially of a random N-mer sequence, or repeats of a random N-mer sequence. In certain embodiments, N can be any integer between 5 and 45. The number of repeats of these N-mer sequences can be 1, 2, 3, 4, 5, 6, 7, 8, 9, or 10.

In certain embodiments, the purification fragment comprises or consists essentially of target-binding oligonucleotides that could specifically bind to a target that is not a nucleotide. In certain embodiments, the target could be proteins, peptides, polysaccharides, or small molecules. In certain embodiments, the purification fragment could be an aptamer that forms a secondary structure specially binds to the target. The target can be one of common affinity ligands used in protein purification, such as biotin, streptavidin, glutathione, chitin, protein A and protein G. A streptavidin-binding RNA aptamer was optimized and used in purification of ribonucleoproteins (An optimized streptavidin-binding RNA aptamer for purification of ribonucleoprotein complexes identifies novel ARE-binding proteins, Nucleic Acids Res. 2014 January; 42 (2): e13). The aptamer that is suitable as the purification fragment provided herein could also be ARC1905, E10030, Pegaptanib sodium (Macugen), EYE001, REG1, NOX-E36, NOX-A12, ARC1779, etc. Available aptamers and their targets have been described in references (e.g. Challenges in delivery of therapeutic genomics and proteomics [M]. Elsevier, 2010.), which could be incorporated herein by reference in their entirety.

In certain embodiments, the purification fragment comprises or consists essentially of a poly(A) tract. In certain embodiments, the poly(A) tract has a length ranging from 15 to 200 nucleotides, from 15 to 150 nucleotides, from 15 to 100 nucleotides, from 15 to 50 nucleotides, or from 15 to 25 nucleotides. In certain embodiments, the poly(A) tract has a length ranging from 15 to 150 nucleotides.

The purification fragment is capable of specifically interacting with or binding to a capturing agent that is external of the RNA transcript. The capturing agent can specifically interact with, bind to, or hybridize to the purification fragment but not to the circular RNA, thereby permitting separation of the circular RNA from the RNA transcript. A capturing agent can be any molecular entity that can specifically interact with or bind to the purification fragment. Examples of capturing agent include, without limitation, a nucleic acid sequence, or nucleic acid binding protein or peptide, or a compound or polymer or any other molecule or material that is capable of binding to the purification fragment.

In certain embodiments, the capturing agent comprises a nucleic acid sequence. In certain embodiments, the capturing agent can comprise a naturally-occurring nucleic acid and/or the analogs, variants, and any mimetics thereof. The capturing agent can have any suitable length of nucleotides (for example 15nt-25nt, or 15nt-40nt, or 15nt-50nt), as long as it can hybridize to the purification fragment.

In certain embodiments, the capturing agent comprises a nucleic acid fragment hybridizable to the purification fragment under stringent conditions. In certain embodiments, the capturing agent comprises or consists essentially of a homo-polymeric or homo-oligomeric sequence of DNA or RNA, including analogs, variants, and any mimetics thereof, complementary to the purification fragment. In certain embodiments, the capturing agent comprises or consists essentially of deoxythymidine oligonucleotide (oligo(dT)), deoxyadenine oligonucleotide (oligo(dA)), oligonucleotide (deoxyadenine: deoxythymidine) (oligo(dA:dT)), deoxycytosine oligonucleotide (oligo(dC)), deoxyguanosine oligonucleotide (oligo(dG)), uridine oligonucleotide (oligo(U)), deoxyuridine oligonucleotide (oligo(dU)) to name just a few.

In certain embodiments, the capturing agent comprises protein, peptide, polysaccharide, or small molecule that is able to bind to the purification fragment as provided herein. In certain embodiments, the capturing agent comprises protein, peptide, polysaccharide, or small molecule that is able to bind to the aptamer fragment comprised in the purification fragment. In certain embodiments, the capturing agent comprises or is a common affinity ligand, such as biotin, streptavidin, glutathione, chitin, protein A and protein G.

In certain embodiments, the capturing agent comprises a nucleic acid fragment at least 80%, 85%, 90%, 95%, or 100% complementary to the nucleotide sequence of the purification fragment. In certain embodiments, the purification fragment comprises or consists essentially of a poly(A) tract, and the capturing agent comprise an oligo(U), oligo(dU), oligo(dT), or any oligonucleotides that is capable of hybridizing to the poly(A) tract. An example of oligo(dT) is a reagent widely used in the art to purify mRNA with a poly(A) tail.

In certain embodiments, the capturing agent can be optionally immobilized on a substrate such as a bead (e.g. magnetic bead) or a chromatographical matrix.

The RNA transcript provided herein comprises at least one purification fragment, for example, 1, or 2 or even more purification fragments.

In certain embodiments, the at least one purification fragment is associated with the first circularization element, or associated with the second circularization element. In certain embodiments, the RNA transcript has two purification fragments that are associated respectively with both the first circularization element and the second circularization element.

In certain embodiments, the at least one purification fragment is: a) located upstream of the first circularization element, b) located downstream of the second circularization element, c) or any combination of a) and b). In certain embodiments, the at least one purification fragment is: a) attached to the 5′ end of the first circularization element, or b) attached to the 3′ end of the second circularization element, or any combination of a) and b).

In certain embodiments, the RNA transcript comprises one purification fragment that is located upstream of the first circularization element, which is also referred to herein as 5′ purification fragment. In certain embodiments, the RNA transcript comprises one purification fragment that is located downstream of the second circularization element, which is also referred to herein as 3′ purification fragment. In certain embodiments, the RNA transcript comprises both the 5′ purification fragment and the 3′ purification fragment, located upstream of the first circularization element and downstream of the second circularization element, respectively.

In certain embodiments, the 5′ purification fragment and the 3′ purification fragment can have identical nucleotide sequence, or alternatively can have different nucleotide sequences.

In certain embodiments, the 5′ purification fragment and the 3′ purification fragment are of the same sequence, and both can bind to the same capturing agent, which allows for separation of the RNA transcript from the circular RNA.

In certain embodiments, the 5′ purification fragment and the 3′ purification fragment have no more than 90%, no more than 80%, no more than 70%, no more than 60%, no more than 50%, or no more than 40% sequence identity. In certain embodiments, the 5′ purification fragment and the 3′ purification fragment have no more than 90%, no more than 80%, no more than 70%, no more than 60%, no more than 50%, or no more than 40% sequence complementarity. In such embodiments, two capturing agents can be used, each specifically bind to one of the 5′ purification fragment and the 3′ purification fragment. The RNA transcript can be separated from the circular RNA using two capturing agents either successively or simultaneously.

### 4. Homology Arm

In certain embodiments, the RNA transcript further comprises at least one homology arm. The homology arm can be located upstream (and optionally adjacent to) of the first circularization element, and such homology arm can be referred to as 5′ homology arm. The homology arm can also be downstream (and optionally adjacent to) of the second circularization element, and such homology arm can be referred to as 3′ homology arm. In certain embodiments, the homology arm can be located upstream (i.e. 5′ homology arm) of the 3′ portion of a self-splicing element and/or downstream (i.e. 3′ homology arm) of the 5′ portion of a self-splicing element.

In certain embodiments, the 5′ homology arm is inserted between the 5′ purification fragment and the first circularization element. In certain embodiments, the 3′ homology arm inserted between the 3′ purification fragment and the second circularization element. In certain embodiments, the 5′ homology arm is located upstream of the 5′ purification fragment and/or the 3′ homology arm is located downstream of the 3′ purification fragment.

In certain embodiments, the RNA transcript comprises both the 5′ homology arm and the 3′ homology arm.

As used herein, a “homology arm” is any contiguous sequence that has at least about 75% (e.g., at least about 80%, at least about 85%, at least about 90%, at least about 95%, about 100%) complementarity to another sequence (e.g. a complementary homology arm) in the same polynucleotide molecule. A homology arm useful in the present disclosure can be of any suitable length, for example, of at least 1 nt (e.g. 10nt, 20nt, 30nt, 40nt, 50nt, 60nt, 70nt, 80nt, 90nt, 100nt, or more) in length, or of up to 250 nt in length. In certain embodiments, the homology arm in the RNA transcript comprises or is an RNA fragment.

In certain embodiments, the RNA transcript has both the 5′ homology arm and the 3′ homology arm, which have at least 75% complementarity. In certain embodiments, each of the homology arms is predicted to have less than 50% (e.g., less than 45%, less than 40%, less than 35%, less than 30%, less than 25%) complementarity with an equal length fragment of an unintended sequence in the RNA transcript (e.g., non-homology arm sequences).

In certain embodiments, the homology arm comprises at least one ALU element derived from ALU repeats. In certain embodiments, the ALU element is associated with the 3′ portion of the self-splicing element and/or the 5′ portion of the self-splicing element (William R. Jeck et al., Circular RNAs are abundant, conserved, and associated with ALU repeats, RNA (2013), 19:141-157).

In certain embodiments, the 5′ homology arm as provided herein is encoded by the nucleic acid sequence comprising SEQ ID NO: 6 (GGGAAGACCCTCGACCGTCGATTGTCCACTGGTC).

In certain embodiments, the 3′ homology arm as provided herein is encoded by the nucleic acid sequence comprising SEQ ID NO: 13 (ACCAGTGGACAATCGACGGATAACAGCATATCTAGG).

### 5. Spacer

In certain embodiments, the RNA transcript further comprises at least one spacer. In certain embodiments, the spacer is located downstream of (and optionally adjacent to) the first circularization element and/or upstream of (and optionally adjacent to) the second circularization element. In certain embodiments, the spacer is located downstream of (and optionally adjacent to) the 3′ portion of a self-splicing element and/or upstream of (and optionally adjacent to) the 5′ portion of a self-splicing element.

In certain embodiments, the spacer is located between the first circularization element (e.g. the 3′ portion of the self-splicing element) and the target sequence, and such spacer can be referred to as 5′ spacer. In certain embodiments, the spacer is located between the target sequence and the second circularization element (e.g. the 5′ portion of the self-splicing element), and such spacer can be referred to as 3′ spacer.

As used herein, a “spacer” refers to a fragment of one or more contiguous nucleotides that intervenes between two other nucleotide sequences, so as to prevent these two other nucleotide sequences from joining together or from interfering with each other's intended biological activity. In certain embodiments, the spacer in the RNA transcript comprises or is an RNA fragment. Without wishing to be bound by any theory, it is believed that inclusion of a spacer between the circularization element and the target sequence is useful to conserve secondary structures present within the circularization element (e.g. intron sequences) that may be important for ribozyme activity, thus allowing higher circularization efficiency than that in DNA constructs without spacers. For example, a spacer can be inserted between for example, the IRES and the circularization element, or between the circularization element and the target coding region or target noncoding region (e.g., UTR region). In certain embodiments, the spacer can be of any suitable length, for example, at least 7 nucleotides long (and optionally no longer than 100 nucleotides).

In certain embodiments, the spacer is located between the circularization element and the IRES sequence. In certain embodiments, the 5′ spacer is located between the first circularization element (e.g. the 3′ portion of the self-splicing element) and the IRES sequence. In certain embodiments, the 3′ spacer is located between the IRES sequence and the second circularization element (e.g. the 5′ portion of the self-splicing element). In certain embodiments, the 5′ spacer is located between the first circularization element and the target protein coding region. In certain embodiments, the 3′ spacer is located between the target protein coding region and the second circularization element. In certain embodiments, the target sequence comprises two complementary spacer sequences. In certain embodiments, the two complementary spacer sequences flank the target sequence provided herein.

In certain embodiments, the spacer contains one or more of the following: a) an unstructured region of at least 5 nt long, b) a region predicted to be complementary to at least 5 nt long to a distal (i.e., non-adjacent) sequence, including another spacer, and/or c) a structured region at least 1 nt long limited in scope to the sequence of the spacer.

In certain embodiments, the spacer is distinct from the purification fragment as provided herein. In certain embodiments, the spacer is not poly(A), poly(U), poly(C), or poly(AC).

In certain embodiments, the spacer is not complementary to (or does not hybridize to) any of the purification fragments. In certain embodiments, the spacer is not complementary to (or does not hybridize to) any of the homology arms.

In certain embodiments, the 5′ spacer as provided herein is encoded by the nucleic acid sequence comprising SEQ ID NO: 8 (TGATCTGAAACCAACTTTATTACTATATTCCCCACAACCCCC).

In certain embodiments, the 3′ spacer as provided herein is encoded by the nucleic acid sequence comprising SEQ ID NO: 11 (GGATCC).

### 6. Exemplary Designs of the Elements Comprised in the RNA Transcript

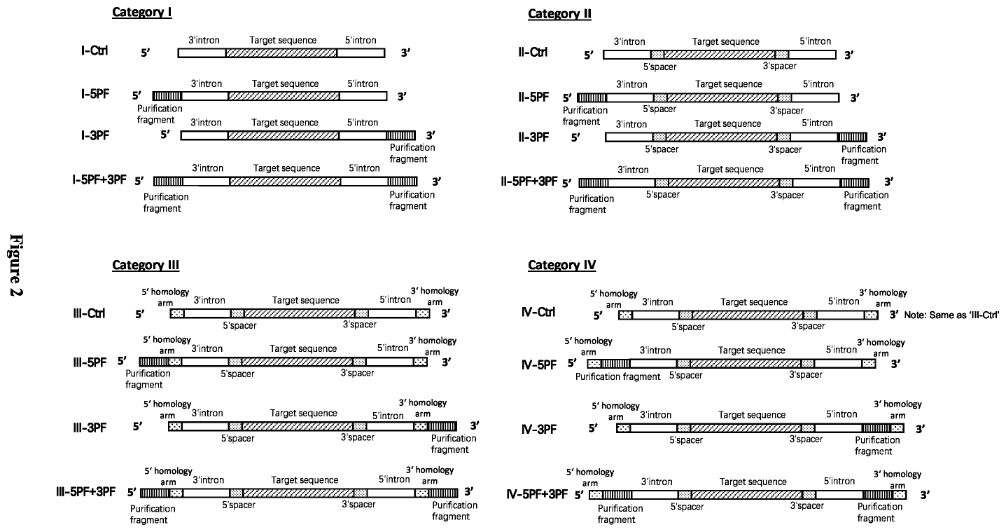

The present disclosure provides exemplary illustrations of the designs of the elements comprised in the RNA transcript provided herein. Schematic drawings demonstrating the exemplary designs are shown in FIG. 2.

In certain embodiments, the RNA transcript comprises from 5′ to 3′, a first circularization element (e.g. 3′ portion of the self-splicing element), a target sequence, and a second circularization element (e.g. 5′ portion of the self-splicing element). These DNA constructs or RNA transcripts have no purification fragments (PFs) are designated as “Ctrl” in FIG. 2. The RNA transcript can further have one purification fragment located either at 5′ end of the first circularization element (designated as 5 PF in FIG. 2), or at the 3′ end of the second circularization element (designated as 3 PF in FIG. 2). Alternatively, the RNA transcript can have two purification fragments located at both the 5′ end of the first circularization element and the 3′ end of the second circularization element, respectively (designated as 5 PF+3 PF in FIG. 2). The two purification fragments provided in the same RNA transcript may or may not be of the same sequence. In particular, the RNA transcript provided herein comprises the following elements operably linked to each other and arranged in the following sequence (from 5′ to 3′):
I-Ctrl: a) 3′ portion of the self-splicing element, b) target sequence, and c) 5′ portion of the self-splicing element;
I-5 PF: a) 5′ purification fragment; b) 3′ portion of the self-splicing element, c) target sequence, and d) 5′ portion of the self-splicing element;
I-3 PF: a) 3′ portion of the self-splicing element, b) target sequence, c) 5′ portion of the self-splicing element, and d) 3′ purification fragment; or
I-5 PF+3 PF: a) 5′ purification fragment; b) 3′ portion of the self-splicing element, c) target sequence, d) 5′ portion of the self-splicing element, and e) 3′ purification fragment.

In certain embodiments, the RNA transcript comprises, from 5′ to 3′ and upstream of the target sequence, a first circularization element (e.g. 3′ portion of the self-splicing element) and, a 5′ spacer. In certain embodiments, the RNA transcript comprises, from 5′ to 3′ and downstream of the target sequence, the 3′ spacer and the second circularization element (e.g. 5′ portion of the self-splicing element). The RNA transcript can have one purification fragment located either at 5′ end of the first circularization element, or at the 3′ end of the second circularization element. Alternatively, the RNA transcript can have two purification fragments located at both the 5′end of the first circularization element and the 3′ end of the second circularization element, respectively. The two purification fragments provided in the same RNA transcript may or may not be of the same sequence. In particular, the RNA transcript provided herein comprises the following elements operably linked to each other and arranged in the following sequence (from 5′ to 3′):
II-Ctrl: a) 3′ portion of the self-splicing element, b) 5′ spacer, c) target sequence, d) 3′ spacer, and e) 5′ portion of the self-splicing element;
II-5 PF: a) 5′ purification fragment; b) 3′ portion of the self-splicing element, c) 5′ spacer, d) target sequence, e) 3′ spacer, and f) 5′ portion of the self-splicing element;
II-3 PF: a) 3′ portion of the self-splicing element, b) 5′ spacer, c) target sequence, d) 3′ spacer, e) 5′ portion of the self-splicing element, and f) 3′ purification fragment; or
II-5 PF+3 PF: a) 5′ purification fragment; b) 3′ portion of the self-splicing element, c) 5′ spacer, d) target sequence, e) 3′ spacer, f) 5′ portion of the self-splicing element, and g) 3′ purification fragment.

In certain embodiments, the RNA transcript comprises, from 5′ to 3′ and upstream of the target sequence, a 5′ homology arm, a first circularization element (e.g. 3′ portion of the self-splicing element), and a 5′ spacer. In certain embodiments, the RNA transcript comprises, from 5′ to 3′ and downstream of the target sequence, a 3′ spacer, a second circularization element (e.g. 5′ portion of the self-splicing element) and a 3′ homology arm. The RNA transcript can have one purification fragment located either at 5′ end of the 5′ homology arm, or at the 3′ end of the 3′ homology arm. Alternatively, the RNA transcript can have two purification fragments located at both the 5′end of the 5′ homology arm and the 3′ end of the 3′ homology arm, respectively. The two purification fragments provided in the same RNA transcript may or may not be of the same sequence. In particular, the RNA transcript provided herein comprises the following elements operably linked to each other and arranged in the following sequence (from 5′ to 3′):
III-Ctrl: a) 5′ homology arm, b) 3′ portion of the self-splicing element, c) 5′ spacer, d) target sequence, e) 3′ spacer, f) 5′ portion of the self-splicing element, and g) 3′ homology arm;
III-5 PF: a) 5′ purification fragment, b) 5′ homology arm, c) 3′ portion of the self-splicing element, d) 5′ spacer, e) target sequence, f) 3′ spacer, g) 5′ portion of the self-splicing element, and h) 3′ homology arm;
III-3 PF: a) 5′ homology arm, b) 3′ portion of the self-splicing element, c) 5′ spacer, d) target sequence, e) 3′ spacer, f) 5′ portion of the self-splicing element, g) 3′ homology arm, and h) 3′ purification fragment; or
III-5 PF+3 PF: a) 5′ purification fragment, b) 5′ homology arm, c) 3′ portion of the self-splicing element, d) 5′ spacer, e) target sequence, f) 3′ spacer, g) 5′ portion of the self-splicing element, h) 3′ homology arm, and i) 3′ purification fragment.

In certain embodiments, the RNA transcript comprises, from 5′ to 3′ and upstream of the target sequence, a 5′ homology arm, a first circularization element (e.g. 3′ portion of the self-splicing element), and a 5′ spacer. In certain embodiments, the target sequence the RNA transcript comprises, from 5′ to 3′ at the downstream of the target sequence, a 3′ spacer, a second circularization element (e.g. 5′ portion of the self-splicing element) and a 3′ homology arm. The RNA transcript can have one purification fragment located either between the 5′ homology arm and the first circularization element, or between the 3′ homology arm and the second circularization element. Alternatively, the RNA transcript can have two purification fragments located between the 5′ homology arm and the first circularization element, and between the 3′ homology arm and the second circularization element, respectively. The two purification fragments provided in the same RNA transcript may or may not be of the same sequence. In particular, the RNA transcript provided herein comprises the following elements operably linked to each other and arranged in the following sequence (from 5′ to 3′):
IV-Ctrl: a) 5′ homology arm, b) 3′ portion of the self-splicing element, c) 5′ spacer, d) target sequence, e) 3′ spacer, f) 5′ portion of the self-splicing element, and g) 3′ homology arm (same as III-Ctrl);
IV-5 PF: a) 5′ homology arm, b) 5′ purification fragment, c) 3′ portion of the self-splicing element, d) 5′ spacer, e) target sequence, f) 3′ spacer, g) 5′ portion of the self-splicing element, and h) 3′ homology arm;
IV-3 PF: a) 5′ homology arm, b) 3′ portion of the self-splicing element, c) 5′ spacer, d) target sequence, e) 3′ spacer, f) 5′ portion of the self-splicing element, g) 3′ purification fragment, and h) 3′ homology arm; or
IV-5 PF+3 PF: a) 5′ homology arm, b) 5′ purification fragment, c) 3′ portion of the self-splicing element, d) 5′ spacer, e) target sequence, f) 3′ spacer, g) 5′ portion of the self-splicing element, h) 3′ purification fragment, and i) 3′ homology arm.

### 7. Circular RNA

In certain embodiments, the RNA transcript provided herein can be circularized to form a circular RNA comprising the target sequence. Circularization can be achieved by intramolecular formation of a 3′,5′-phosphodiester bond, which requires close proximity of the 3′- and 5′-terminus of the linear RNA transcript.

In certain embodiments, circularization can be achieved through a back-splicing reaction, or exon skipping. In certain embodiment, the first circularization element comprises a 3′ portion of a self-splicing element comprising a 3′ proximal fragment of an intron portion and the adjacent exon portion of at least 1 nucleotide in length. In certain embodiment, the second circularization element comprises a 5′ portion of a self-splicing element comprising of a 5′ proximal fragment of an intron and the adjacent exon portion of at least 1 nucleotide in length. This circularization process involves joining the 3′-tail of a downstream exon portion to the 5′-head of an exon portion of at least 1 nucleotide in length that is normally upstream. This is facilitated by the addition of suitable enzymes or factors that promote the formation of RNA-RNA bonds. In certain embodiments, the resulting circular RNA molecules could contain the exon portion of the first circularization element and the exon portion of the second circularization element, but are free of the intron portions of the first circularization element and the second circularization element, respectively. Other splicing methods to provide circular RNA are known in the art, for example, see, circular RNAs Biogenesis and Functions, edited by Junjie Xiao. Other suitable methods can be used to produce circular RNA, see for example, Sonja Petkovic and Sabine Müller, RNA circularization strategies in vivo and in vitro, Nucleic Acids Research, 2015, Vol. 43, No. 4, 2454-2465.

In certain embodiments, the resulting circular RNA molecules may further comprise at least one spacer. In certain embodiments, the spacer is located between the exon portion of the first circularization element and the target sequence, and such spacer can be referred to as 5′ spacer. In certain embodiments, the spacer is located between the target sequence and the exon portion of the second circularization element, and such spacer can be referred to as 3′ spacer.

In certain embodiment, the resulting circular RNA comprises the target sequence that comprises an internal ribosome entry site (IRES) operably linked to the target protein coding region.

### DNA Constructs as Vectors for Further Construction of DNA Constructs

In another aspect, the present disclosure provides DNA constructs comprising a multi-cloning site and DNA encoding for at least one purification fragment. Such a DNA construct allows for insertion of one or more target sequences provided herein, and/or the first circularization element and/or the second circularization element in the multi-cloning site, such that, the inserted target sequence and/or the circularization element will be operably linked to the purification fragment contained in the DNA construct. It should be understood that in the context of DNA constructs, the terms like “circularization element”, “target sequence”, “purification fragment”, “homology arm” and “spacer” are intended to mean DNA sequences that encode for the corresponding elements (i.e. circularization element, target sequence, purification fragment, homology arm and spacer) of the RNA transcript as described herein. For example, when used in the context of DNA construct, the term “circularization element” can refer to the DNA encoding for the circularization element in the RNA transcript, the term “target sequence” can refer to the DNA encoding for the target sequence in the RNA transcript, the term “purification fragment” can refer to the DNA encoding for the purification fragment in the RNA transcript, the term “homology arm” can refer to the DNA encoding for the homology arm in the RNA transcript, and the term “spacer” can refer to the DNA encoding for the spacer in the RNA transcript.

As used herein, the term “code” or “encode” refers to the inherent property of specific sequences of nucleotides in a polynucleotide, such as a DNA, or an RNA, to serve as templates for synthesis of other polymers and macromolecules in in-vivo or in-vitro biological processes having either a defined sequence of nucleotides or a defined sequence of amino acids and the biological properties resulting therefrom. For example, a DNA encodes for an RNA transcript if the transcription of said DNA produces said RNA transcript in a suitable in-vivo or in-vitro biological system, an RNA transcript encodes for a protein if the translation of said RNA transcript produces said protein in a suitable in-vivo or in-vitro biological system, and a DNA encodes for a protein if the transcription and translation of said DNA produces said protein in a suitable in-vivo or in-vitro biological system. Both the coding strand (sense strand), the nucleic acid sequence of which is identical to the RNA transcript sequence and is usually provided in sequence listings, and the non-coding strand (non-sense strand), used as the template for transcription of a DNA, can be referred to as encoding the protein or other product of said DNA.

Such a DNA construct can be used as a vector or a tool for construction of the DNA construct provided herein, i.e., the DNA construct encoding for the RNA transcript provided herein, that is capable of forming a circular RNA comprising the target sequence.

The present disclosure also provides a DNA construct comprising the following elements operably linked to each other and arranged in 5′ to 3′ sequence:
a) a first purification fragment, optionally a first poly(A) tract, and
b) a multi-cloning site comprising or consisting essentially of one or more cloning sites for inserting a target sequence.

In certain embodiments, the DNA construct further comprises a second purification fragment downstream of the multi-cloning site.

In certain embodiments, the DNA construct further comprises a first circularization element between the first purification fragment and the multi-cloning site. In certain embodiments, the DNA construct further comprises a second circularization element downstream of the multi-cloning site, or between the multi-cloning site and the second purification fragment.

In certain embodiments, the second purification fragment comprises a second poly(A) tract.

In certain embodiments, the DNA construct further comprises an RNA polymerase promoter at the upstream of the first purification fragment.

The present disclosure further provides a DNA construct comprising the following elements operably linked to each other and arranged in the following sequence:
a) an RNA polymerase promoter,
b) a first circularization element,
c) a multi-cloning site, and
d) a second circularization element,
wherein the DNA construct further comprises at least one purification fragment that is downstream of the RNA polymerase promoter.

In certain embodiments, one of the purification fragments is located upstream of the first circularization element, and/or downstream of the second circularization element.

As used herein, the term “multi-cloning site” or “MCS” refers to a short segment of DNA which contains multiple (up to ˜20) restriction sites and is a standard element of engineered plasmids and other vectors. Restriction sites within an MCS are typically unique, which is present only once within a vector, and can therefore be used to insert a sequence of interest into the vector. Furthermore, expression vectors are often designed so that the MCS can insert the protein encoding sequence in the correct reading frame by choosing the correct insertion site, and/or the user can select the reading frame by choice of MCS, which are often available in all three frames.

### Methods for Circular RNA Preparation

The present disclosure provides a method of producing the RNA transcript as provided herein, the method comprising transcribing from the DNA construct provided herein, thereby obtaining the RNA transcript provided herein.

In another aspect, the RNA transcript provided herein may be produced by tagging the purification fragment at an RNA level to a precursor. The present disclosure also provides a method of producing the RNA transcript as provided herein, the method comprising:
a) providing a precursor RNA which differs from the RNA transcript provided herein in lacking the purification fragment, and
b) adding the purification fragment to the precursor RNA, thereby obtaining said RNA transcript.

In certain embodiments, the precursor RNA differs from the RNA transcript provided herein only in lacking the purification fragment.

In certain embodiments, the purification fragment is added to: a) a position located upstream of the first circularization element, b) a position located downstream of the second circularization element, c) or any combination of a) and b).

In certain embodiments, the purification fragment is: a) attached to the 5′ end of the first circularization element, or b) attached to the 3′ end of the second circularization element, c) or any combination of a) and b).

The present disclosure further provides a method of producing a circular RNA, the method comprising:
a) providing said RNA transcript, and
b) allowing self-circularization of the RNA transcript to form the circular RNA.

In certain embodiments, the RNA transcript in step a) has been enriched using the capturing agent disclosed herein. The capturing agent can specifically bind to the purification fragment present in the RNA transcript.

In certain embodiments, the method further comprises purifying the circular RNA from the product obtained in step b) by the capturing agent provided herein. In certain embodiments, the capturing agent specifically binds to the purification fragment in the RNA transcript and optionally in a by-product but absent in the circular RNA.

The term “by-product” used herein refers to any intermediate fragment that occurs in the process of producing the circular RNA. A by-product is not neither identical to the RNA transcript nor to the circular RNA. The by-product may contain partial length of the RNA transcript provided herein. The by-product may contain at least part of the purification fragment which may be captured by the capturing agent.

In certain embodiments, the capturing agent used to separate the RNA transcript from the circular RNA comprises a nucleic acid fragment hybridizable to the purification fragment under stringent conditions. In certain embodiments, the capturing agent comprises a nucleic acid fragment at least 80%, 85%, 90%, 95%, or 100% complementary to the nucleotide sequence of the purification fragment.

In certain embodiments, the capturing agent is immobilized. In certain embodiments, the RNA transcript of step b) undergoes self-circularization either in solution or on solid matrix where the capturing agent is immobilized. In certain embodiments, the purification fragment comprises a poly(A) tract and the capturing agent comprises oligo(dT). Other oligonucleotides can also be used as capturing agents as long as they are complementary to the PF in the RNA transcripts, such as using Oligo-(AT) n as capturing agent for PF of poly(AU) or Oligo-(CT) n as capturing agent for PF of poly(AG). In certain embodiments, the purification fragment can bind to capturing agents comprises proteins, peptides, polysaccharides, or small molecules. In certain embodiments, the capturing agent comprises or is a common affinity ligand, such as biotin, streptavidin, glutathione, chitin, protein A and protein G, which binds to PFs of RNA aptamers of unique ligand-binding structures.

In certain embodiments, the RNA transcript in step a) is transcribed from the DNA construct provided herein. In certain embodiments, the method further comprises, before step a), transcribing from the DNA construct provided herein to produce the RNA transcript provided herein.

### Compositions

The present disclosure provides a composition of circular RNA produced using the method provided herein. The present disclosure also provides a composition comprising the DNA construct provided herein, or the RNA transcript provided herein.

In certain embodiments, the present disclosure provides pharmaceutical compositions comprising an effective amount of a circular RNA described herein and a pharmaceutically acceptable excipient. Pharmaceutical compositions of the present disclosure may comprise a circular RNA as described herein, in combination with one or more pharmaceutically or physiologically acceptable carriers, excipients or diluents. In some embodiments, pharmaceutical compositions of the present disclosure may comprise a circular RNA expressing cell, e.g., a plurality of circular RNA-expressing cells, as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject.

In some embodiments, a circular RNA as described herein may be used in combination with other known agents and therapies. In further embodiments, a composition described herein may be used in a treatment regimen in combination with surgery, radiation, chemotherapy, antibodies, or other agents.

### Kits

The present disclosure provides a kit comprising said DNA construct. The present disclosure also provides a kit for producing said RNA transcript, comprising a reagent useful for adding the purification fragment to the precursor of the RNA transcript.

In certain embodiments, the kit further comprises a capturing agent that specifically binds to the purification fragment present in the RNA transcript but absent in the circular RNA. In certain embodiments, the capturing agent used to separate the RNA transcript from the circular RNA comprises a nucleic acid fragment hybridizable to the purification fragment under stringent conditions. In certain embodiments, the capturing agent is immobilized. In certain embodiments, the purification fragment comprises a poly(A) tract and the capturing agent comprises oligo(dT).

The kit provided herein may also include one or more transfection reagents to facilitate delivery of circular RNAs (or recombinant nucleic acids encoding them) to cells. Such kits may also include components that preserve the polynucleotides or that protect against their degradation. Such components may be RNase-free or protect against RNases.

Such kits generally will comprise, in suitable means, distinct containers for each individual reagent or solution. The kit may comprise one or more containers holding the circular RNAs, or recombinant polynucleotides encoding them, and other agents. Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

The kit can further comprise a container comprising a pharmaceutically acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

### EXAMPLES

### Example 1. The DNA Constructs (Plasmids)

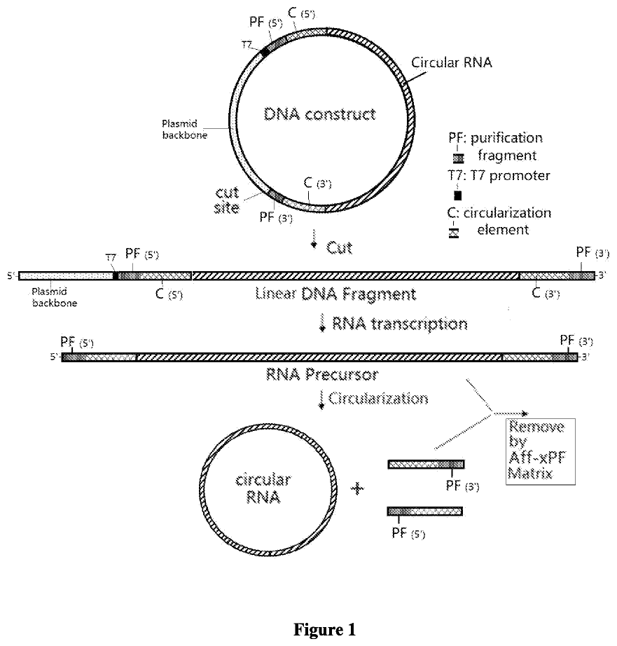

In general, DNA constructs illustrated in FIG. 1 can be made to generate circular RNA according to the process indicated by arrows in FIG. 1. Each construct has a plasmid backbone (which may include Ori and antibiotic resistance gene), a promoter for RNA polymerase (e.g. T7 promoter), and a DNA sequence for an RNA precursor. The DNA constructs can be transcribed to RNA precursors (or RNA transcripts) as exemplified in FIG. 2 in a total of four categories (Category I, Category II, Category III, and Category IV), including: I-Ctrl, I-5 PF, I-3 PF, I-5 PF+3 PF, II-Ctrl, II-5 PF, II-3 PF, II-5 PF+3 PF, III-Ctrl, III-5 PF, III-3 PF, III-5 PF+3 PF, IV-Ctrl, IV-5 PF, IV-3 PF, and IV-5 PF+3 PF.

Details of the RNA transcripts are shown in FIG. 2 and summarized below. All the RNA transcripts designated as “Ctrl” were used as negative controls, as they do not have any purification fragment.

| RNA | |
|---|---|
| transcripts | Elements (from 5′ to 3′) |
| I-Ctrl | a) 3′ portion of the self-splicing element, b) target sequence, and c) 5′ |
| | portion of the self-splicing element |
| I-5PF | a) 5′ purification fragment; b) 3′ portion of the self-splicing element, c) |
| | target sequence, and d) 5′ portion of the self-splicing element |
| I-3PF | a) 3′ portion of the self-splicing element, b) target sequence, c) 5′ portion |
| | of the self-splicing element, and d) 3′ purification fragment |
| I-5PF + | a) 5′ purification fragment; b) 3′ portion of the self-splicing element, c) |
| 3PF | target sequence, d) 5′ portion of the self-splicing element, and e) 3′ |
| | purification fragment |
| II-Ctrl | a) 3′ portion of the self-splicing element, b) 5′ spacer, c) target sequence, |
| | d) 3′ spacer, and e) 5′ portion of the self-splicing element |
| II-5PF | a) 5′ purification fragment; b) 3′ portion of the self-splicing element, c) |
| | 5′ spacer, d) target sequence, e) 3′ spacer, and f) 5′ portion of the self- |
| | splicing element |
| II-3PF | a) 3′ portion of the self-splicing element, b) 5′ spacer, c) target sequence, |
| | d) 3′ spacer, e) 5′ portion of the self-splicing element, and f) 3′ |
| | purification fragment |
| II-5PF + | a) 5′ purification fragment; b) 3′ portion of the self-splicing element, c) |
| 3PF | 5′ spacer, d) target sequence, e) 3′ spacer, f) 5′ portion of the self-splicing |
| | element, and g) 3′ purification fragment |
| III-Ctrl | a) 5′ homology arm, b) 3′ portion of the self-splicing element, c) 5′ spacer, |
| | d) target sequence, e) 3′ spacer, f) 5′ portion of the self-splicing element, |
| | and g) 3′ homology arm; |
| III-5PF | a) 5′ purification fragment, b) 5′ homology arm, c) 3′ portion of the self- |
| | splicing element, d) 5′ spacer, e) target sequence, f) 3′ spacer, g) 5′ portion |
| | of the self-splicing element, and h) 3′ homology arm; |
| III-3PF | a) 5′ homology arm, b) 3′ portion of the self-splicing element, c) 5′ spacer, |
| | d) target sequence, e) 3′ spacer, f) 5′ portion of the self-splicing element, |
| | g) 3′ homology arm, and h) 3′ purification fragment |
| III-5PF + | a) 5′ purification fragment, b) 5′ homology arm, c) 3′ portion of the self- |
| 3PF | splicing element, d) 5′ spacer, e) target sequence, f) 3′ spacer, g) 5′ portion |
| | of the self-splicing element, h) 3′ homology arm, and i) 3′ purification |
| | fragment |
| IV-Ctrl | a) 5′ homology arm, b) 3′ portion of the self-splicing element, c) 5′ spacer, |
| | d) target sequence, e) 3′ spacer, f) 5′ portion of the self-splicing element, |
| | and g) 3′ homology arm (same as III-Ctrl) |
| IV-5PF | a) 5′ homology arm, b) 5′ purification fragment, c) 3′ portion of the self- |
| | splicing element, d) 5′ spacer, e) target sequence, f) 3′ spacer, g) 5′ portion |
| | of the self-splicing element, and h) 3′ homology arm |
| IV-3PF | a) 5′ homology arm, b) 3′ portion of the self-splicing element, c) 5′ spacer, |
| | d) target sequence, e) 3′ spacer, f) 5′ portion of the self-splicing element, |
| | g) 3′ purification fragment, and h) 3′ homology arm |
| IV-5PF + | a) 5′ homology arm, b) 5′ purification fragment, c) 3′ portion of the self- |
| 3PF | splicing element, d) 5′ spacer, e) target sequence, f) 3′ spacer, g) 5′ portion |
| | of the self-splicing element, h) 3′ purification fragment, and i) 3′ |
| | homology arm |

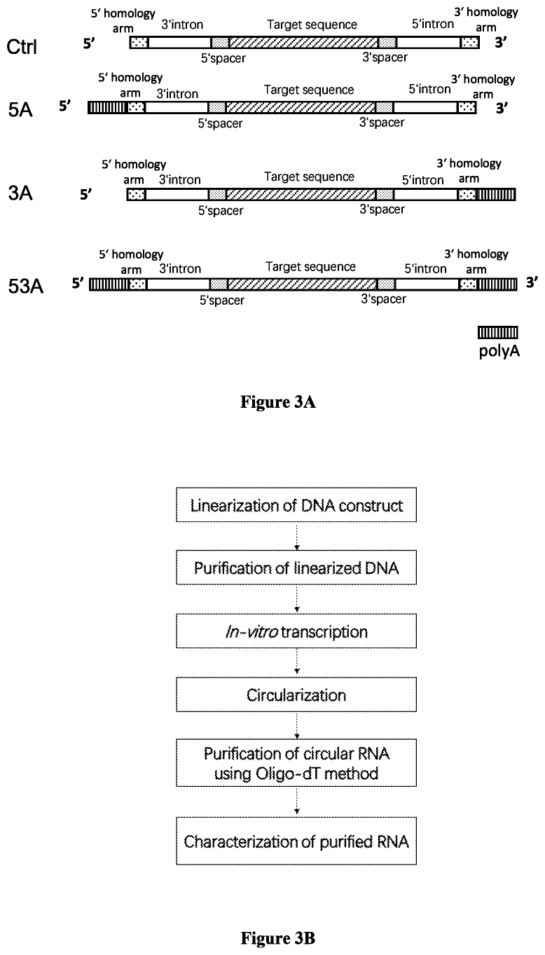

The RNA precursor contains a target sequence (e.g., a gene encoding GFP protein) to be circularized, circularization element(s) (e.g., Group I intron self-splicing elements) and purification fragment(s) (e.g., poly-A tracts as shown in FIG. 3). The purification fragments are at or near either or both ends of the transcribed RNA. In category III and category IV, the constructs further have homology fragments (i.e., homology arms, to enable the close proximity of circularization ends). In some of these constructs, the purification fragments are at the joint or joints of homology fragments and circularization elements. The purification fragments are not part of the final circular products. The purification fragment is selected to have a length within 20-200 nucleotides. In category IV, the location of the purification fragment is between the circularization element and homology arm, while in category III, the purification fragment lies at the end of homology arms. In Category I and II, the location of the purification fragments (e.g., poly(A)) is designed at 3′ end of the RNA precursor, 5′ end of the RNA precursor or both ends, as shown in FIG. 1 and FIG. 2.

Exemplified DNA sequences encoding for each element of the target sequence, 3′ portion of the self-splicing element (i.e., circularization element, designated as “3′ intron” in FIG. 2), 5′ portion of the self-splicing element (i.e., circularization element, designated as “5′ intron” in FIG. 2), 5′ purification fragment, 3′ purification fragment, 5′ homology arm, 3′ homology arm, and IRES are provided as below (from 5′ to 3′).

| Element | DNA encoding sequence |
|---|---|
| 5′ purification | SEQ ID NO: 5: |
| fragment | AAAAAAAAAAAAAAAAAAAAAAAAAAAAAAAAAAAAAAAA |
| 5′ homology | SEQ ID NO.: 6: |
| arm | GGGAAGACCCTCGACCGTCGATTGTCCACTGGTC |
| 3′ portion of the | SEQ ID NO: 7: |
| self-splicing | AACAATAGATGACTTACAACTAATCGGAAGGTGCAGAGACTCGACGGG |
| element | AGCTACCCTAACGTCAAGACGAGGGTAAAGAGAGAGTCCAATTCTCAA |
| (designated as | AGCCAATAGGCAGTAGCGAAAGCTGCAAGAGAATGAAAATCCGTTGAC |
| “3′ intron″ in | CTTAAACGGTCGTGTGGGTTCAAGTCCCTCCACCCCCA |
| FIG. 2) | |
| 5′ spacer | SEQ ID NO: 8: |
| | TGATCTGAAACCAACTTTATTACTATATTCCCCACAACCCCC |
| IRES | SEQ ID NO: 9: |
| | TTAAAACAGCCTGTGGGTTGATCCCACCCACAGGCCCATTGGGCGCTA |
| | GCACTCTGGTATCACGGTACCTTTGTGCGCCTGTTTTATACCCCCTCCCC |
| | CAACTGTAACTTAGAAGTAACACACACCGATCAACAGTCAGCGTGGCA |
| | CACCAGCCACGTTTTGATCAAGCACTTCTGTTACCCCGGACTGAGTATC |
| | AATAGACTGCTCACGCGGTTGAAGGAGAAAGCGTTCGTTATCCGGCCA |
| | ACTACTTCGAAAAACCTAGTAACACCGTGGAAGTTGCAGAGTGTTTCG |
| | CTCAGCACTACCCCAGTGTAGATCAGGTCGATGAGTCACCGCATTCCCC |
| | ACGGGCGACCGTGGCGGTGGCTGCGTTGGCGGCCTGCCCATGGGGAAA |
| | CCCATGGGACGCTCTAATACAGACATGGTGCGAAGAGTCTATTGAGCT |
| | AGTTGGTAGTCCTCCGGCCCCTGAATGCGGCTAATCCTAACTGCGGAGC |
| | ACACACCCTCAAGCCAGAGGGCAGTGTGTCGTAACGGGCAACTCTGCA |
| | GCGGAACCGACTACTTTGGGTGTCCGTGTTTCATTTTATTCCTATACTGG |
| | CTGCTTATGGTGACAATTGAGAGATCGTTACCATATAGCTATTGGATTG |
| | GCCATCCGGTGACTAATAGAGCTATTATATATCCCTTTGTTGGGTT |
| eGFP coding | SEQ ID NO: 10: |
| sequence | ATGGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGC |
| | GAGGGCGAGGGCGATGCCACCTACGGCAAGCTGACCCTGAAGTTCATC |
| | TGCACCACCGGCAAGCTGCCCGTGCCCTGGCCCACCCTCGTGACCACCC |
| | TGACCTACGGCGTGCAGTGCTTCAGCCGCTACCCCGACCACATGAAGC |
| | AGCACGACTTCTTCAAGTCCGCCATGCCCGAAGGCTACGTCCAGGAGC |
| | GCACCATCTTCTTCAAGGACGACGGCAACTACAAGACCCGCGCCGAGG |
| | TGAAGTTCGAGGGCGACACCCTGGTGAACCGCATCGAGCTGAAGGGCA |
| | TCGACTTCAAGGAGGACGGCAACATCCTGGGGCACAAGCTGGAGTACA |
| | ACTACAACAGCCACAACGTCTATATCATGGCCGACAAGCAGAAGAACG |
| | GCATCAAGGTGAACTTCAAGATCCGCCACAACATCGAGGACGGCAGCG |
| | TGCAGCTCGCCGACCACTACCAGCAGAACACCCCCATCGGCGACGGCC |
| | CCGTGCTGCTGCCCGACAACCACTACCTGAGCACCCAGTCCGCCCTGAG |
| | CAAAGACCCCAACGAGAAGCGCGATCACATGGTCCTGCTGGAGTTCGT |
| | GACCGCCGCCGGGATCACTCTCGGCATGGACGAGCTGTACAAGTAG |
| 3′ spacer | SEQ ID NO: 11: |
| | GGATCC |
| 5′ portion of the | SEQ ID NO: 12: |
| self-splicing | AGACGCTACGGACTTAAATAATTGAGCCTTAAAGAAGAAATTCTTTAA |
| element | GTGGATGCTCTCAAACTCAGGGAAACCTAAATCTAGTTATAGACAAGG |
| (designated as | CAATCCTGAGCCAAGCCGAAGTAGTAATTAGTAAG |
| “5′ intron″ in | |
| FIG. 2) | |
| 3′ homology | SEQ ID NO: 13: |
| arm | ACCAGTGGACAATCGACGGATAACAGCATATCTAGG |
| 3′ purification | SEQ ID NO: 14: |
| fragment | AAAAAAAAAAAAAAAAAAAAAAAAAAAAAAAAAAAAAAAAAAAAAA |
| | AAAAAAAAAAAAAAAAAAAAAAAAAAAAAAAAAA |

To exemplify the DNA constructs, the Category III is further illustrated with poly-A tract as the PF, as shown in FIG. 3A. The DNA sequence encoding the full length of the RNA precursors of Category III are provided as below (from 5′ to 3′).

Accordingly, plasmids carrying these sequences (SEQ ID NO: 1-4) were constructed and verified by DNA sequencing analysis.

| RNA precursor | DNA encoding sequence |
|---|---|
| III-Ctrl-eGFP | SEQ ID NO: 1: |
| | TAATACGACTCACTATAGGGTCGACGTCGACGGGAAGACCCTCGACCG |
| | TCGATTGTCCACTGGTCAACAATAGATGACTTACAACTAATCGGAAGGT |
| | GCAGAGACTCGACGGGAGCTACCCTAACGTCAAGACGAGGGTAAAGA |
| | GAGAGTCCAATTCTCAAAGCCAATAGGCAGTAGCGAAAGCTGCAAGAG |
| | AATGAAAATCCGTTGACCTTAAACGGTCGTGTGGGTTCAAGTCCCTCCA |
| | CCCCCATGATCTGAAACCAACTTTATTACTATATTCCCCACAACCCCCA |
| | GTACTTTAAAACAGCCTGTGGGTTGATCCCACCCACAGGCCCATTGGGC |
| | GCTAGCACTCTGGTATCACGGTACCTTTGTGCGCCTGTTTTATACCCCCT |
| | CCCCCAACTGTAACTTAGAAGTAACACACACCGATCAACAGTCAGCGT |
| | GGCACACCAGCCACGTTTTGATCAAGCACTTCTGTTACCCCGGACTGAG |
| | TATCAATAGACTGCTCACGCGGTTGAAGGAGAAAGCGTTCGTTATCCG |
| | GCCAACTACTTCGAAAAACCTAGTAACACCGTGGAAGTTGCAGAGTGT |
| | TTCGCTCAGCACTACCCCAGTGTAGATCAGGTCGATGAGTCACCGCATT |
| | CCCCACGGGCGACCGTGGCGGTGGCTGCGTTGGCGGCCTGCCCATGGG |
| | GAAACCCATGGGACGCTCTAATACAGACATGGTGCGAAGAGTCTATTG |
| | AGCTAGTTGGTAGTCCTCCGGCCCCTGAATGCGGCTAATCCTAACTGCG |
| | GAGCACACACCCTCAAGCCAGAGGGCAGTGTGTCGTAACGGGCAACTC |
| | TGCAGCGGAACCGACTACTTTGGGTGTCCGTGTTTCATTTTATTCCTATA |
| | CTGGCTGCTTATGGTGACAATTGAGAGATCGTTACCATATAGCTATTGG |
| | ATTGGCCATCCGGTGACTAATAGAGCTATTATATATCCCTTTGTTGGGT |
| | TACTAGTGATAATAGTGCCACCATGGTGAGCAAGGGCGAGGAGCTGTT |
| | CACCGGGGTGGTGCCCATCCTGGTCGAGCTGGACGGCGACGTAAACGG |
| | CCACAAGTTCAGCGTGTCCGGCGAGGGCGAGGGCGATGCCACCTACGG |
| | CAAGCTGACCCTGAAGTTCATCTGCACCACCGGCAAGCTGCCCGTGCCC |
| | TGGCCCACCCTCGTGACCACCCTGACCTACGGCGTGCAGTGCTTCAGCC |
| | GCTACCCCGACCACATGAAGCAGCACGACTTCTTCAAGTCCGCCATGCC |
| | CGAAGGCTACGTCCAGGAGCGCACCATCTTCTTCAAGGACGACGGCAA |
| | CTACAAGACCCGCGCCGAGGTGAAGTTCGAGGGCGACACCCTGGTGAA |
| | CCGCATCGAGCTGAAGGGCATCGACTTCAAGGAGGACGGCAACATCCT |
| | GGGGCACAAGCTGGAGTACAACTACAACAGCCACAACGTCTATATCAT |
| | GGCCGACAAGCAGAAGAACGGCATCAAGGTGAACTTCAAGATCCGCCA |
| | CAACATCGAGGACGGCAGCGTGCAGCTCGCCGACCACTACCAGCAGAA |
| | CACCCCCATCGGCGACGGCCCCGTGCTGCTGCCCGACAACCACTACCTG |
| | AGCACCCAGTCCGCCCTGAGCAAAGACCCCAACGAGAAGCGCGATCAC |
| | ATGGTCCTGCTGGAGTTCGTGACCGCCGCCGGGATCACTCTCGGCATGG |
| | ACGAGCTGTACAAGTAGGGATCCAGACGCTACGGACTTAAATAATTGA |
| | GCCTTAAAGAAGAAATTCTTTAAGTGGATGCTCTCAAACTCAGGGAAA |
| | CCTAAATCTAGTTATAGACAAGGCAATCCTGAGCCAAGCCGAAGTAGT |
| | AATTAGTAAGACCAGTGGACAATCGACGGATAACAGCATATCTAGGGA |
| | ATTC |
| III-5A-eGFP | SEQ ID NO: 2: |
| | TAATACGACTCACTATAGGGTCGACAAAAAAAAAAAAAAAAAAAAAA |
| | AAAAAAAAAAAAAAAAAAGTCGACGGGAAGACCCTCGACCGTCGATT |
| | GTCCACTGGTCAACAATAGATGACTTACAACTAATCGGAAGGTGCAGA |
| | GACTCGACGGGAGCTACCCTAACGTCAAGACGAGGGTAAAGAGAGAG |
| | TCCAATTCTCAAAGCCAATAGGCAGTAGCGAAAGCTGCAAGAGAATGA |
| | AAATCCGTTGACCTTAAACGGTCGTGTGGGTTCAAGTCCCTCCACCCCC |
| | ATGATCTGAAACCAACTTTATTACTATATTCCCCACAACCCCCAGTACT |
| | TTAAAACAGCCTGTGGGTTGATCCCACCCACAGGCCCATTGGGCGCTA |
| | GCACTCTGGTATCACGGTACCTTTGTGCGCCTGTTTTATACCCCCTCCCC |
| | CAACTGTAACTTAGAAGTAACACACACCGATCAACAGTCAGCGTGGCA |
| | CACCAGCCACGTTTTGATCAAGCACTTCTGTTACCCCGGACTGAGTATC |
| | AATAGACTGCTCACGCGGTTGAAGGAGAAAGCGTTCGTTATCCGGCCA |
| | ACTACTTCGAAAAACCTAGTAACACCGTGGAAGTTGCAGAGTGTTTCG |
| | CTCAGCACTACCCCAGTGTAGATCAGGTCGATGAGTCACCGCATTCCCC |
| | ACGGGCGACCGTGGCGGTGGCTGCGTTGGCGGCCTGCCCATGGGGAAA |
| | CCCATGGGACGCTCTAATACAGACATGGTGCGAAGAGTCTATTGAGCT |
| | AGTTGGTAGTCCTCCGGCCCCTGAATGCGGCTAATCCTAACTGCGGAGC |
| | ACACACCCTCAAGCCAGAGGGCAGTGTGTCGTAACGGGCAACTCTGCA |
| | GCGGAACCGACTACTTTGGGTGTCCGTGTTTCATTTTATTCCTATACTGG |
| | CTGCTTATGGTGACAATTGAGAGATCGTTACCATATAGCTATTGGATTG |
| | GCCATCCGGTGACTAATAGAGCTATTATATATCCCTTTGTTGGGTTACT |
| | AGTGATAATAGTGCCACCATGGTGAGCAAGGGCGAGGAGCTGTTCACC |
| | GGGGTGGTGCCCATCCTGGTCGAGCTGGACGGCGACGTAAACGGCCAC |
| | AAGTTCAGCGTGTCCGGCGAGGGCGAGGGCGATGCCACCTACGGCAAG |
| | CTGACCCTGAAGTTCATCTGCACCACCGGCAAGCTGCCCGTGCCCTGGC |
| | CCACCCTCGTGACCACCCTGACCTACGGCGTGCAGTGCTTCAGCCGCTA |
| | CCCCGACCACATGAAGCAGCACGACTTCTTCAAGTCCGCCATGCCCGA |
| | AGGCTACGTCCAGGAGCGCACCATCTTCTTCAAGGACGACGGCAACTA |
| | CAAGACCCGCGCCGAGGTGAAGTTCGAGGGCGACACCCTGGTGAACCG |
| | CATCGAGCTGAAGGGCATCGACTTCAAGGAGGACGGCAACATCCTGGG |
| | GCACAAGCTGGAGTACAACTACAACAGCCACAACGTCTATATCATGGC |
| | CGACAAGCAGAAGAACGGCATCAAGGTGAACTTCAAGATCCGCCACAA |
| | CATCGAGGACGGCAGCGTGCAGCTCGCCGACCACTACCAGCAGAACAC |
| | CCCCATCGGCGACGGCCCCGTGCTGCTGCCCGACAACCACTACCTGAG |
| | CACCCAGTCCGCCCTGAGCAAAGACCCCAACGAGAAGCGCGATCACAT |
| | GGTCCTGCTGGAGTTCGTGACCGCCGCCGGGATCACTCTCGGCATGGAC |
| | GAGCTGTACAAGTAGGGATCCAGACGCTACGGACTTAAATAATTGAGC |
| | CTTAAAGAAGAAATTCTTTAAGTGGATGCTCTCAAACTCAGGGAAACC |
| | TAAATCTAGTTATAGACAAGGCAATCCTGAGCCAAGCCGAAGTAGTAA |
| | TTAGTAAGACCAGTGGACAATCGACGGATAACAGCATATCTAGGGAAT |
| | TC |
| III-3A-eGFP | SEQ ID NO: 3: |
| | TAATACGACTCACTATAGGGTCGACGTCGACGGGAAGACCCTCGACCG |
| | TCGATTGTCCACTGGTCAACAATAGATGACTTACAACTAATCGGAAGGT |
| | GCAGAGACTCGACGGGAGCTACCCTAACGTCAAGACGAGGGTAAAGA |
| | GAGAGTCCAATTCTCAAAGCCAATAGGCAGTAGCGAAAGCTGCAAGAG |
| | AATGAAAATCCGTTGACCTTAAACGGTCGTGTGGGTTCAAGTCCCTCCA |
| | CCCCCATGATCTGAAACCAACTTTATTACTATATTCCCCACAACCCCCA |
| | GTACTTTAAAACAGCCTGTGGGTTGATCCCACCCACAGGCCCATTGGGC |
| | GCTAGCACTCTGGTATCACGGTACCTTTGTGCGCCTGTTTTATACCCCCT |
| | CCCCCAACTGTAACTTAGAAGTAACACACACCGATCAACAGTCAGCGT |
| | GGCACACCAGCCACGTTTTGATCAAGCACTTCTGTTACCCCGGACTGAG |
| | TATCAATAGACTGCTCACGCGGTTGAAGGAGAAAGCGTTCGTTATCCG |
| | GCCAACTACTTCGAAAAACCTAGTAACACCGTGGAAGTTGCAGAGTGT |
| | TTCGCTCAGCACTACCCCAGTGTAGATCAGGTCGATGAGTCACCGCATT |
| | CCCCACGGGCGACCGTGGCGGTGGCTGCGTTGGCGGCCTGCCCATGGG |
| | GAAACCCATGGGACGCTCTAATACAGACATGGTGCGAAGAGTCTATTG |
| | AGCTAGTTGGTAGTCCTCCGGCCCCTGAATGCGGCTAATCCTAACTGCG |
| | GAGCACACACCCTCAAGCCAGAGGGCAGTGTGTCGTAACGGGCAACTC |
| | TGCAGCGGAACCGACTACTTTGGGTGTCCGTGTTTCATTTTATTCCTATA |
| | CTGGCTGCTTATGGTGACAATTGAGAGATCGTTACCATATAGCTATTGG |
| | ATTGGCCATCCGGTGACTAATAGAGCTATTATATATCCCTTTGTTGGGT |
| | TACTAGTGATAATAGTGCCACCATGGTGAGCAAGGGCGAGGAGCTGTT |
| | CACCGGGGTGGTGCCCATCCTGGTCGAGCTGGACGGCGACGTAAACGG |
| | CCACAAGTTCAGCGTGTCCGGCGAGGGCGAGGGCGATGCCACCTACGG |
| | CAAGCTGACCCTGAAGTTCATCTGCACCACCGGCAAGCTGCCCGTGCCC |
| | TGGCCCACCCTCGTGACCACCCTGACCTACGGCGTGCAGTGCTTCAGCC |
| | GCTACCCCGACCACATGAAGCAGCACGACTTCTTCAAGTCCGCCATGCC |
| | CGAAGGCTACGTCCAGGAGCGCACCATCTTCTTCAAGGACGACGGCAA |
| | CTACAAGACCCGCGCCGAGGTGAAGTTCGAGGGCGACACCCTGGTGAA |
| | CCGCATCGAGCTGAAGGGCATCGACTTCAAGGAGGACGGCAACATCCT |
| | GGGGCACAAGCTGGAGTACAACTACAACAGCCACAACGTCTATATCAT |
| | GGCCGACAAGCAGAAGAACGGCATCAAGGTGAACTTCAAGATCCGCCA |
| | CAACATCGAGGACGGCAGCGTGCAGCTCGCCGACCACTACCAGCAGAA |
| | CACCCCCATCGGCGACGGCCCCGTGCTGCTGCCCGACAACCACTACCTG |
| | AGCACCCAGTCCGCCCTGAGCAAAGACCCCAACGAGAAGCGCGATCAC |
| | ATGGTCCTGCTGGAGTTCGTGACCGCCGCCGGGATCACTCTCGGCATGG |
| | ACGAGCTGTACAAGTAGGGATCCAGACGCTACGGACTTAAATAATTGA |
| | GCCTTAAAGAAGAAATTCTTTAAGTGGATGCTCTCAAACTCAGGGAAA |
| | CCTAAATCTAGTTATAGACAAGGCAATCCTGAGCCAAGCCGAAGTAGT |
| | AATTAGTAAGACCAGTGGACAATCGACGGATAACAGCATATCTAGGGA |
| | ATTCAAAAAAAAAAAAAAAAAAAAAAAAAAAAAAAAAAAAAAAAAA |
| | AAAAAAAAAAAAAAAAAAAAAAAAAAAAAAAAAAAAAA |
| III-35A-eGFP | SEQ ID NO: 4: |
| | TAATACGACTCACTATAGGGTCGACAAAAAAAAAAAAAAAAAAAAAA |
| | AAAAAAAAAAAAAAAAAAGTCGACGGGAAGACCCTCGACCGTCGATT |
| | GTCCACTGGTCAACAATAGATGACTTACAACTAATCGGAAGGTGCAGA |
| | GACTCGACGGGAGCTACCCTAACGTCAAGACGAGGGTAAAGAGAGAG |
| | TCCAATTCTCAAAGCCAATAGGCAGTAGCGAAAGCTGCAAGAGAATGA |
| | AAATCCGTTGACCTTAAACGGTCGTGTGGGTTCAAGTCCCTCCACCCCC |
| | ATGATCTGAAACCAACTTTATTACTATATTCCCCACAACCCCCAGTACT |
| | TTAAAACAGCCTGTGGGTTGATCCCACCCACAGGCCCATTGGGCGCTA |
| | GCACTCTGGTATCACGGTACCTTTGTGCGCCTGTTTTATACCCCCTCCCC |
| | CAACTGTAACTTAGAAGTAACACACACCGATCAACAGTCAGCGTGGCA |
| | CACCAGCCACGTTTTGATCAAGCACTTCTGTTACCCCGGACTGAGTATC |
| | AATAGACTGCTCACGCGGTTGAAGGAGAAAGCGTTCGTTATCCGGCCA |
| | ACTACTTCGAAAAACCTAGTAACACCGTGGAAGTTGCAGAGTGTTTCG |
| | CTCAGCACTACCCCAGTGTAGATCAGGTCGATGAGTCACCGCATTCCCC |
| | ACGGGCGACCGTGGCGGTGGCTGCGTTGGCGGCCTGCCCATGGGGAAA |
| | CCCATGGGACGCTCTAATACAGACATGGTGCGAAGAGTCTATTGAGCT |
| | AGTTGGTAGTCCTCCGGCCCCTGAATGCGGCTAATCCTAACTGCGGAGC |
| | ACACACCCTCAAGCCAGAGGGCAGTGTGTCGTAACGGGCAACTCTGCA |
| | GCGGAACCGACTACTTTGGGTGTCCGTGTTTCATTTTATTCCTATACTGG |
| | CTGCTTATGGTGACAATTGAGAGATCGTTACCATATAGCTATTGGATTG |
| | GCCATCCGGTGACTAATAGAGCTATTATATATCCCTTTGTTGGGTTACT |
| | AGTGATAATAGTGCCACCATGGTGAGCAAGGGCGAGGAGCTGTTCACC |
| | GGGGTGGTGCCCATCCTGGTCGAGCTGGACGGCGACGTAAACGGCCAC |
| | AAGTTCAGCGTGTCCGGCGAGGGCGAGGGCGATGCCACCTACGGCAAG |
| | CTGACCCTGAAGTTCATCTGCACCACCGGCAAGCTGCCCGTGCCCTGGC |
| | CCACCCTCGTGACCACCCTGACCTACGGCGTGCAGTGCTTCAGCCGCTA |
| | CCCCGACCACATGAAGCAGCACGACTTCTTCAAGTCCGCCATGCCCGA |
| | AGGCTACGTCCAGGAGCGCACCATCTTCTTCAAGGACGACGGCAACTA |
| | CAAGACCCGCGCCGAGGTGAAGTTCGAGGGCGACACCCTGGTGAACCG |
| | CATCGAGCTGAAGGGCATCGACTTCAAGGAGGACGGCAACATCCTGGG |
| | GCACAAGCTGGAGTACAACTACAACAGCCACAACGTCTATATCATGGC |
| | CGACAAGCAGAAGAACGGCATCAAGGTGAACTTCAAGATCCGCCACAA |
| | CATCGAGGACGGCAGCGTGCAGCTCGCCGACCACTACCAGCAGAACAC |
| | CCCCATCGGCGACGGCCCCGTGCTGCTGCCCGACAACCACTACCTGAG |
| | CACCCAGTCCGCCCTGAGCAAAGACCCCAACGAGAAGCGCGATCACAT |
| | GGTCCTGCTGGAGTTCGTGACCGCCGCCGGGATCACTCTCGGCATGGAC |
| | GAGCTGTACAAGTAGGGATCCAGACGCTACGGACTTAAATAATTGAGC |
| | CTTAAAGAAGAAATTCTTTAAGTGGATGCTCTCAAACTCAGGGAAACC |
| | TAAATCTAGTTATAGACAAGGCAATCCTGAGCCAAGCCGAAGTAGTAA |
| | TTAGTAAGACCAGTGGACAATCGACGGATAACAGCATATCTAGGGAAT |
| | TCAAAAAAAAAAAAAAAAAAAAAAAAAAAAAAAAAAAAAAAAAAAA |
| | AAAAAAAAAAAAAAAAAAAAAAAAAAAAAAAAAAAA |

### Example 2. Purification and Characterization of Circular RNA

The DNA constructs made in accordance with Example 1 were linearized and in vitro transcribed, circularized and purified in accordance with the experimental workflow provided below. Both circularization efficiency and purification efficiency were assessed and compared to demonstrate the benefits of PFs.

### 2.1 Experimental Workflow

As shown in FIG. 3B, the purification and characterization of circular RNA were carried out according to steps including linearization of DNA construct, purification of linearized DNA, in vitro transcription, circularization, purification of circular RNA using Oligo-dT method, and characterization of purified RNA.

### 2.2 Experimental Procedures and Results

### Preparation of Plasmid DNA.

DNA constructs (plasmids) as constructed in Example 1 (including those as shown in FIG. 3A, Ctrl, 5A, 3A and 53A) were synthesized and cloned into an E. coli strain for plasmid replication. The plasmids were extracted and purified before linearized by enzymatic digestion.

The plasmid DNAs were digested with a restriction enzyme HindIII (NEB, R0104L) under the condition of 37° C. for 1-4 h. Then the linearized plasmid DNAs were purified using a QIAquick Gel Extraction Kit.

Linear RNA transcripts were synthesized by in vitro transcription from the linearized plasmid DNA template using T7 RNA polymerase in the presence of NTPs, Murine RNase inhibitor and Pyrophosphatase (Inorganic) at 37° C. for 30-120 min.

To produce circular RNA, additional circularization reaction buffer containing 50 mM Tris-HCl, pH7.4, 10 mM MgCl₂ and 2 mM GTP was added into the mixture of in-vitro transcription and further incubated at 55° C. for 15-30 min. The generation of circular RNA was detected by agarose gel electrophoresis.

The obtained circular RNA was then purified by passing the RNA mixture through an oligo-dT agent. RNA fragments carrying poly-A tract(s) would bind/retain on the Oligo-dT agent, while the circRNA product would be in the flowthrough, and hence gets purified. For bench scale purification, magnetic beads conjugated with Oligo (dT) 25 (Ambion, Cat. No.: 61002) were used according to the manufacturer's protocols. Alternatively, a column made of Oligo-dT resins was also used for the purification of circular RNA on an AKTA equipment (Cytiva). To illustrate the purification process, the purification was carried out with these steps: binding, washing, and elution of bound RNAs.

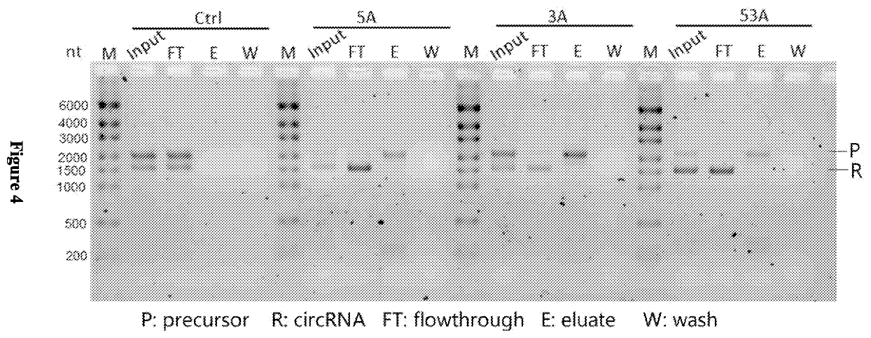

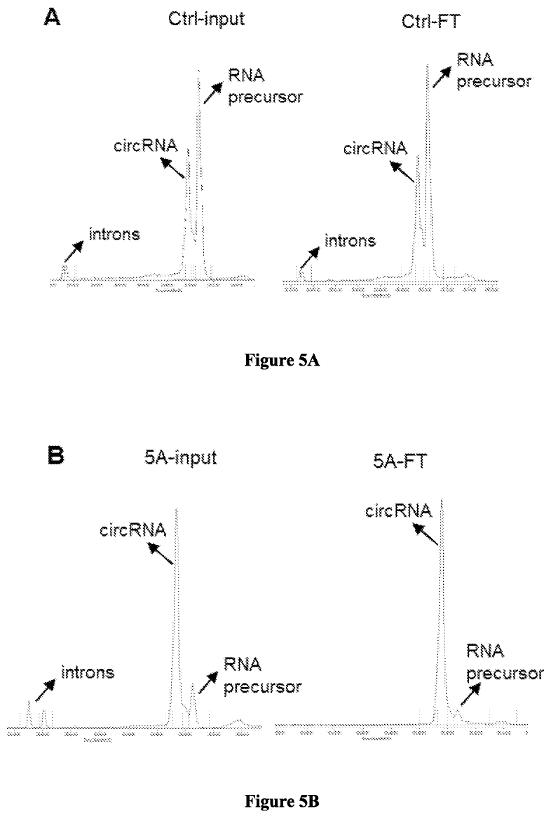

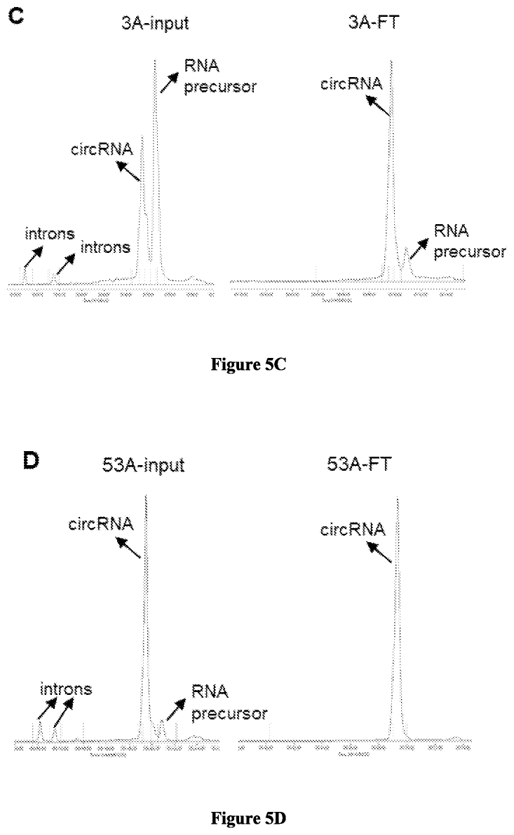

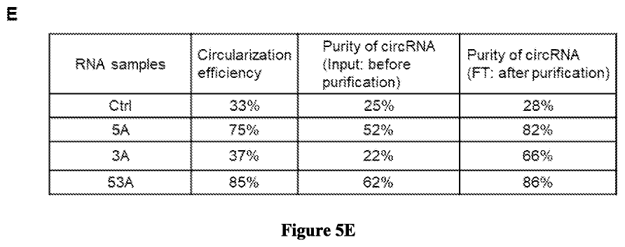

Fractions were collected from the following purification steps: FT (flowthrough) from the binding step, W from the washing step, and E (eluate) from the elution step. These fractions together with RNA mixture from the circularization step (denoted as Input in FIGS. 4 and 5A-5E) were analyzed on agarose gels (FIG. 4) and by capillary electrophoresis (CE) (FIGS. 5A-5E). Circularization efficiency and circRNA purity were calculated based on CE results (FIG. 5E).

FIGS. 4 and 5A-5E show the fractions following the purification steps for circular RNA products made from Category III constructs, i.e., those as shown in FIG. 3A (Ctrl, 5A, 3A and 53A). The bands on the lanes of Input (which denotes RNA mixture from the circularization step), FT (which denotes the flowthrough from the binding step) and E fractions (which denotes eluate from the elution step) showed that PF-containing RNA fragments were effectively bound to the capturing agent, Oligo-dT magnetic beads, removed from the circRNA products and eluted with an elution buffer (FIG. 4).

Clearly from these analyses, adding a poly-A tract at either end of RNA precursor or at each end of RNA precursor did not negatively affect circularization efficiency. Instead, as compared to Ctrl, the presence of the PF at 5′ end of the RNA precursor surprisingly and significantly increased circularization efficiency, from 33% for Ctrl to 75% and 85% for 5A and 53A products, respectively (FIG. 5A-5E). In addition, as summarized in FIG. 5E, Ctrl product exhibited an increase of circRNA purity from 25% to 28% before and after purification. In contrast, 5A product exhibited a growth of circRNA purity from 52% to 82%, 3A product exhibited a growth from 22% to 66% and 53A product exhibited a growth from 62% to 86%, respectively before and after purification. In relative to the construct in absence of the purification fragment as disclosed herein, the three constructs comprising one or two poly-A tract(s) as the purification fragment all demonstrated remarkably higher effectiveness in circRNA purification. Following the method of producing the circRNA as disclosed herein, the purified circRNA achieved a purity of up to almost 90%.

Fractions following the purification steps for circular RNA products made from Category I, Category II and Category IV constructs were also analyzed using the similar process. The presence of poly-A tracts as PFs dramatically facilitated purification of resulting circRNAs, resulting highly purified circRNAs as expected.

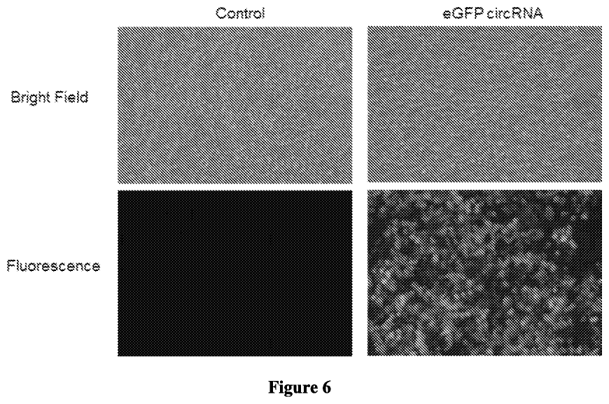

To test the functionality of the circRNA products, a transfection experiment was carried out. The purified GFP circRNA products were transfected into HEK 293T cells by lipofectamine. After incubation for two days, fluorescent images were taken on a microscope under bright field and with GFP settings (FIG. 6). The strong fluorescent field clearly showed the expression of GFP proteins, demonstrating the functionality of the synthesized GFP circRNA.

### Example 3. Synthesis, Purification and Characterization of Circular RNAs

Plasmids carrying Category III elements as illustrated in FIG. 2 with an aptamer as the purification fragment were also constructed according to the methods as described in Examples 1 and 2, and verified by DNA sequencing analysis.

The DNA constructs were linearized and in vitro transcribed, circularized and purified in accordance with the experimental workflow as provided in Example 2. [The Target that is capable of specifically binding to the aptamer] was used as the capturing agent. Both circularization efficiency and purification efficiency were assessed and compared to demonstrate the benefits of PFs.

Similarly, fractions were collected from the following purification steps: FT (flowthrough) from the binding step, W from washing step, and E (eluate) from elution step. These fractions together with RNA mixture from the circularization step (Input) were analyzed on agarose gels and by capillary electrophoresis (CE). Circularization efficiencies and circRNA purity were calculated based on CE results.

Experimental data showed that comprising an aptamer as the purification fragment achieved a similar performance to comprising poly-A tract as the purification fragment. The presence of the aptamer as PF facilitated the purification process, yielding circRNA products of high purity. The purified circRNA products also showed the desired function of the target sequence contained therein.

1. A DNA construct for making a circular RNA, the DNA construct comprising an RNA polymerase promoter operably linked to a sequence coding for an RNA transcript capable of producing a circular RNA, said RNA transcript comprises the following elements operably linked to each other and arranged in the following sequence:
a) a first circularization element,
b) a target sequence, and
c) a second circularization element,
wherein the RNA transcript is capable of forming the circular RNA comprising the target sequence; and
wherein the RNA transcript further comprises at least one purification fragment that is absent in the circular RNA.

2. The DNA construct of claim 1, wherein the RNA polymerase promoter is an RNA polymerase promotor derived from T7 virus, T6 virus, SP6 virus, T3 virus, or T4 virus.

3. An in vitro transcribed RNA transcript, said RNA transcript comprises the following elements operably linked to each other and arranged in the following sequence:
a) a first circularization element,
b) a target sequence, and
c) a second circularization element,
wherein the RNA transcript is capable of forming a circular RNA comprising the target sequence; and
wherein the RNA transcript further comprises at least one purification fragment that is absent in the circular RNA.

4. The DNA construct of claim 1 or 2, or the RNA transcript of claim 3, wherein the at least one purification fragment is associated with the first circularization element, or associated with the second circularization element.

5. The DNA construct of claim 1 or 2, or the RNA transcript of claim 3, wherein the RNA transcript has two purification fragments that are associated respectively with both the first circularization element and the second circularization element.

6. The DNA construct of any one of claims 1-2, and 4-5, or the RNA transcript of any one of claims 3-5, wherein the at least one purification fragment is: a) located upstream of the first circularization element (i.e. 5′ purification fragment), b) located downstream of the second circularization element (i.e. 3′ purification fragment), c) or any combination of a) and b).

7. The DNA construct of any one of claims 1-2, and 4-6, or the RNA transcript of any one of claims 3-6, wherein the at least one purification fragment is: a) attached to the 5′ end of the first circularization element (i.e. 5′ purification fragment), or b) attached to the 3′ end of the second circularization element (i.e. 3′ purification fragment), or any combination of a) and b).

8. The DNA construct of any one of claims 6-7 or the RNA transcript of any one of claims 6-7, wherein the RNA transcript has both the 5′ purification fragment and the 3′ purification fragment, and wherein the 5′ purification fragment and the 3′ purification fragment are either identical or different in nucleotide sequence.

9. The DNA construct of any one of claims 1-2, and 4-8 or the RNA transcript of any one of claims 3-7, wherein each of at least one of the purification fragment has no more than 90% (or no more than 80%, no more than 70%, no more than 60%, no more than 50%) sequence identity to the circular RNA in a given length of 15-20 nucleotides.

10. The DNA construct of any one of claims 1-2, and 4-9 or the RNA transcript of any one of claims 3-9, wherein the purification fragment comprises a poly(A) tract, poly(T) tract, poly(U) tract, poly(C) tract, poly(G) tract, poly(AC) tract, poly(AG) tract, poly(CT) tract, poly(CU) tract, poly(AT) tract, or poly(AU) tract.

11. The DNA construct of claim 10, or the RNA transcript of claim 10, wherein the purification fragment (e.g. the poly(A) tract) has a length ranging from 15 to 200 nucleotides, optionally from 15 to 150 nucleotides.

12. The DNA construct of any one of claims 1-2, and 4-11 or the RNA transcript of any one of claims 3-11, wherein the first circularization element and the second circularization element can be derived from a self-splicing system.

13. The DNA construct of claim 12 or the RNA transcript of claim 12, wherein the first circularization element comprises a 3′ portion of a self-splicing element comprising a 3′ splice site, and the second circularization element comprises a 5′ portion of the self-splicing element comprising a 5′ splice site.

14. The DNA construct of claim 13 or the RNA transcript of claim 13, wherein the self-splicing element is a Group I intron, a Group II intron, or a hairpin ribozyme.

15. The DNA construct of claim 14, or the RNA transcript of claim 14, wherein the Group I intron is derived from phage T4 thymidylate synthase (td) gene, Cyanobacterium Anabaena sp. pre-tRNA-Leu gene, or Tetrahymena gene.

16. The DNA construct of claim 14, or the RNA transcript of claim 14, wherein the Group II intron is derived from L. lactis Ll.LtrB gene, or derived from yeast.

17. The DNA construct of claim 14, or the RNA transcript of claim 14, wherein the hairpin ribozyme is derived from bacteria, eukaryotes, or plant virus RNA satellites (e.g., tobacco ringspot virus (TRSV) satellite RNA, chicory yellow mottle virus (sCYMV) satellite RNA, or arabis mosaic virus (sARMV) satellite RNA).

18. The DNA construct of any one of claims 1-2 and 4-17 or the RNA transcript of any one of claims 3-17, wherein the target sequence comprises a target protein coding region.

19. The DNA construct of claim 18, or the RNA transcript of claim 18, wherein the target protein is a therapeutic protein (e.g. an antibody, cytokine, peptide hormone, etc.), a prophylactic protein (e.g. a protein vaccine, etc.), a nuclease (e.g. Cas protein, recombinase, etc.), a receptor (e.g. chimeric antigen receptor, etc.).

20. The DNA construct of claim 18 or 19, or the RNA transcript of claim 18 or 19, wherein the target sequence further comprises an internal ribosome entry site (IRES) operably linked to the target protein coding region, optionally the IRES is operably linked upstream and/or downstream of the target protein coding region.

21. The DNA construct of claim 20, or the RNA transcript of claim 20, wherein the IRES is selected from an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picoma-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Salivirus, Cosavirus, Parechovirus, Human UNR, Mouse UtrA, Human VEGF-A, Human XIAP, Drosophila hairless, S. cerevisiae TFIID, S. cerevisiae YAP1, Human c-src, Human FGF-1, Simian picomavirus, Turnip crinkle virus, an aptamer to eaF4G, Coxsackievirus B3 (CVB3) or Coxsackievirus A (CVB1/2).

22. The DNA construct of anyone of claims 18-21, or the RNA transcript of anyone of claims 18-21, wherein the target sequence further comprises a 5′ Untranslated Region (UTR) and/or a 3′ UTR operably linked to the target protein coding region.

23. The DNA construct of any one of claims 1-2 and 4-17 or the RNA transcript of any one of claims 3-17, wherein the target sequence comprises a biologically active RNA or a precursor of the biologically active RNA.

24. The DNA construct of claim 23, or the RNA transcript of claim 23, wherein the biologically active RNA comprises a short hairpin RNA, transfer RNA (tRNA), short interfering RNA, microRNA, or guide RNA.

25. The DNA construct of any one of claims 1-2 and 4-24 or the RNA transcript of any one of claims 3-23, wherein the RNA transcript further comprises at least one homology arm, optionally the homology arm is located upstream (i.e. 5′ homology arm) of the first circularization element and/or downstream (i.e. 3′ homology arm) of the second circularization element.

26. The DNA construct of claim 25 or the RNA transcript of claim 25, wherein the homology arm comprises at least one ALU element derived from ALU repeats.

27. The DNA construct of claim 24 or 25 or the RNA transcript of claim 24 or 25, wherein the homology arm is:
a) inserted between the 5′ purification fragment and the first circularization element and/or inserted between the 3′ purification fragment and the second circularization element; or
b) located upstream of the 5′ purification fragment and/or downstream of the 3′ purification fragment.

28. The DNA construct of any one of claims 1-2 and 4-27 or the RNA transcript of any one of claims 3-27, wherein the RNA transcript further comprises at least one spacer, optionally the spacer is located between the first circularization element (e.g. the 3′ portion of the self-splicing element) and the target sequence, and/or between the target sequence and the second circularization element (e.g. the 5′ portion of the self-splicing element).

29. The DNA construct of claim 28 or the RNA transcript of claim 28, wherein the spacer is:
a) located between the first circularization element (e.g. the 3′ portion of the self-splicing element) and the IRES sequence, and/or between the target sequence and the second circularization element (e.g. the 5′ portion of the self-splicing element);
b) located between the first circularization element (e.g. the 3′ portion of the self-splicing element) and the target protein coding region, and/or between the target protein coding region and the second circularization element (e.g. the 5′ portion of the self-splicing element).

30. The DNA construct of claim 28 or 29 or the RNA transcript of claim 28 or 29, wherein the at least one spacer sequences comprise two complementary spacer sequences flanking the target sequence.

31. A DNA construct comprising the following elements operably linked to each other and arranged in 5′ to 3′ sequence:
a) a first purification fragment, optionally a first poly(A) tract, and
b) a multi-cloning site comprising or consisting essentially of one or more cloning sites for inserting a target sequence.

32. The DNA construct of claim 31, further comprising, a second purification fragment downstream of the multi-cloning site.

33. The DNA construct of claim 31 or 32, further comprising, a first circularization element between the first purification fragment and the multi-cloning site, and/or a second circularization element downstream of the multi-cloning site, or between the multi-cloning site and the second purification fragment.

34. The DNA construct of any of claims 31-33, further comprising, an RNA polymerase promoter at the upstream of the first purification fragment.

35. A DNA construct comprising the following elements operably linked to each other and arranged in the following sequence:
a) an RNA polymerase promoter,
b) a first circularization element,
c) a multi-cloning site, and
d) a second circularization element,
wherein the DNA construct further comprises at least one purification fragment that is downstream of the RNA polymerase promoter.

36. The DNA construct of claim 35, wherein one of the purification fragments is located upstream of the first circularization element, and/or downstream of the second circularization element.

37. A method of producing the RNA transcript of any one of claims 3-30, the method comprising: transcribing from the DNA construct of any one of claims 1-2 and 4-30, thereby obtaining the RNA transcript of any one of claims 3-30.

38. A method of producing the RNA transcript of any one of claims 3-30, the method comprising:
a) providing a precursor RNA which differs from the RNA transcript in lacking the purification fragment, optionally the precursor RNA differs from the RNA transcript only in lacking the purification fragment, and
b) adding the purification fragment to the precursor RNA, thereby obtaining the RNA transcript of any one of claims 3-30.

39. The method of claim 38, wherein the purification fragment is added to: a) a position located upstream of the first circularization element, b) a position located downstream of the second circularization element, c) or any combination of a) and b).

40. The method of claim 38, wherein the purification fragment is: a) attached to the 5′ end of the first circularization element, or b) attached to the 3′ end of the second circularization element, c) or any combination of a) and b).

41. A method of producing a circular RNA, the method comprising:
a) providing the RNA transcript of any one of claims 3-30, and
b) allowing self-circularization of the RNA transcript to form the circular RNA.

42. The method of claim 41, wherein the RNA transcript in step a) has been enriched using a capturing agent that specifically binds to the purification fragment present in the RNA transcript.

43. The method of claim 41 or 42, wherein the method further comprises purifying the circular RNA from the product obtained in step b) by a capturing agent, wherein the capturing agent specifically binds to the purification fragment in the RNA transcript and optionally in a by-product but absent in the circular RNA.

44. The method of claim 42 or 43, wherein the capturing agent comprises a nucleic acid fragment hybridizable to the purification fragment under stringent conditions, optionally the capturing agent comprises a nucleic acid fragment at least 80%, 85%, 90%, 95%, or 100% complementary to the nucleotide sequence of the purification fragment.

45. The method of claim 44, wherein the capturing agent is immobilized.

46. The method of any one of claims 41-45, wherein the RNA transcript in step b) undergoes self-circularization either in solution or on solid matrix where the capturing agent is immobilized.

47. The method of any one of claims 41-46, wherein the purification fragment comprises a poly(A) tract and the capturing agent comprises deoxythymidine oligonucleotide (oligo(dT)).

48. The method of any one of claims 41-47, wherein the RNA transcript in step a) is transcribed from the DNA construct of any one of claims 1-2 and 4-30.

49. The method of claim 41, wherein the method further comprises, before step a), transcribing from the DNA construct of any one of claims 1-2 and 4-30 to produce the RNA transcript of any one of claims 3-30.

50. The method of claim 41, wherein the RNA transcript in step a) is obtained by the method of any one of claims 38-40.

51. A composition of circular RNA produced using the method of any one of claims 41-50.

52. A composition comprising the DNA construct of any one of claims 1-2 and 4-30, or the RNA transcript of any one of claims 3-30.

53. A kit comprising the DNA construct of any one of claims 1-2 and 4-36.

54. A kit for producing the RNA transcript as defined in any one of claims 3-30, comprising a reagent useful for adding the purification fragment to the precursor of the RNA transcript as defined in claim 38.

55. The kit of claim 53 or 54, wherein the kit further comprises a capturing agent that specifically binds to the purification fragment that is present in the RNA transcript but absent in the circular RNA.

56. The kit of claim 53 or 54, wherein the purification fragment comprises a poly(A) tract.

57. The kit of claim 53 or 54, wherein the capturing agent comprises oligo(dT).
